(19) United States
(12) Patent Application Publication
(10) Pub. No.: US 2025/0065077 A1
(43) Pub. Date: Feb. 27, 2025

(54) MEDICAL ACCESS SHEATHS

(71) Applicant: EDWARDS LIFESCIENCES CORPORATION, Irvine, CA (US)

(72) Inventors: Michael G. Valdez, Riverside, CA (US); Daniel James Murray, Orange, CA (US); Hien T. Nguyen, Aliso Viejo, CA (US); Steven Park, Santa Ana, CA (US)

(21) Appl. No.: 18/947,203

(22) Filed: Nov. 14, 2024

Related U.S. Application Data
(63) Continuation of application No. PCT/US2023/022869, filed on May 19, 2023.
(60) Provisional application No. 63/345,885, filed on May 25, 2022.

(51) Int. Cl. A61M 25/00

(52) U.S. Cl. CPC A61M 25/0026; A61M 25/0074

(57) ABSTRACT

A tapered medical access sheath can comprise an access sheath hub and an access sheath shaft extending distally from the access sheath hub. The access sheath shaft can comprise a first delivery lumen extending from a proximal end of the access sheath shaft to a side opening on a distal portion of the access sheath shaft, the first delivery lumen having a width configured to slidably receive a distal portion of a medical device disposed around a guidewire. The access sheath shaft can comprise a tapered distal end portion disposed distally of the side opening. A recess can extend along a longitudinal dimension of the tapered distal end portion from a proximal end to a distal end of the tapered distal end portion. The recess can be in fluid communication with the first delivery lumen and have a width smaller than the diameter of the first delivery lumen.

### CROSS-REFERENCE TO RELATED APPLICATION(S)

This application is a continuation of International Patent Application No. PCT/US2023/022869, filed May 19, 2023, which claims the benefit of U.S. Provisional Patent Application No. 63/345,885, filed May 25, 2022, the disclosures of which are hereby expressly incorporated by reference herein in their entireties for all purposes.

### BACKGROUND

The present disclosure generally relates to the field of minimally invasive transcatheter delivery of medical devices. Minimally invasive transcatheter medical procedures can comprise use of medical sheaths for delivery of medical devices. Minimally invasive transcatheter procedures can be used to treat a variety of conditions, including conditions of the heart.

### SUMMARY

Described herein are methods, systems, and devices related to tapered access sheaths configured to receive at least a portion of a medical device to facilitate transcatheter delivery of the medical device through tortuous anatomical pathways in minimally invasive procedures, providing mechanical strength to prevent or reduce damage to the medical device. The tapered access sheath can comprise an access sheath shaft with a first delivery lumen extending from a proximal end to a side opening on a distal portion of the access sheath shaft. A tapered distal end portion can be distal of the side opening. A recess having the same or similar orientation as the side opening can extend distally from the first delivery lumen through the tapered distal end portion to a distal end of the access sheath shaft.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

For purposes of summarizing the disclosure, certain aspects, advantages and novel features have been described herein. It is to be understood that not necessarily all such advantages may be achieved in accordance with any particular example. Thus, the disclosed examples may be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes and should in no way be interpreted as limiting the scope of the inventions. In addition, various features of different disclosed examples can be combined to form additional examples, which are part of this disclosure. Throughout the drawings, reference numbers may be reused to indicate correspondence between reference elements. However, it should be understood that the use of similar reference numbers in connection with multiple drawings does not necessarily imply similarity between respective examples associated therewith. Furthermore, it should be understood that the features of the respective drawings are not necessarily drawn to scale, and the illustrated sizes thereof are presented for the purpose of illustration of inventive aspects thereof. Generally, certain of the illustrated features may be relatively smaller than as illustrated in some examples or configurations.

FIG. 1 shows a tapered access sheath comprising a portion inserted through an introducer sheath, where the introducer sheath comprises an introducer sheath shaft positioned into a femoral artery in accordance with one or more examples.

FIGS. 2A, 2B, 2C and 2D provide perspective, top-down, side, and longitudinal cross-sectional views, respectively, of the tapered access sheath described with reference to FIG. 1.

FIGS. 3A, 3B and 3C provide longitudinal cross-sectional views showing delivery of a medical device through the tapered access sheath described with reference to FIGS. 1 and 2A through 2D in accordance with one or more examples.

FIGS. 4A, 4B, 4C and 4D provide perspective, top-down, side, and longitudinal cross-sectional views, respectively, of a tapered access sheath comprising a recess configured to receive a proximal portion of a medical device in accordance with one or more examples.

FIGS. 5A, 5B, 5C and 5D provide longitudinal cross-sectional views showing delivery of a medical device through the tapered access sheath described with reference to FIGS. 4A through 4D in accordance with one or more examples.

FIGS. 6A and 6B provide perspective and side views, respectively, of a tapered access sheath comprising an access sheath shaft with a protrusion extending along at least a portion of a sidewall portion defining a side opening on the access sheath shaft in accordance with one or more examples.

FIGS. 7A, 7B, 7C and 7D provide longitudinal cross-sectional views showing delivery of a medical device through the tapered access sheath described with reference to FIGS. 6A and 6B in accordance with one or more examples.

FIG. 8 is a process flow diagram illustrating a process for delivering a first and a second medical device to a target location using a medical delivery sheath assembly as described herein in accordance with one or more examples.

### DETAILED DESCRIPTION

The headings provided herein are for convenience only and do not necessarily affect the scope or meaning of the claimed invention.

The present disclosure provides systems, devices, and methods relating to tapered access sheaths configured to receive at least a portion of a medical device for transcatheter delivery of the medical device. The tapered access sheath can comprise an access sheath shaft with a first delivery lumen extending from a proximal end of the access sheath shaft to a side opening on a distal portion of the access sheath shaft. The medical device can be slidably received by the first delivery lumen, for example being advanced through the first delivery lumen and out of the side opening. A tapered distal end portion can be disposed distally of the side opening to facilitate navigation of the tapered access sheath through anatomical pathways. A recess having the same or similar orientation as the side opening can be in fluid communication with and extend distally from the first delivery lumen through the tapered distal end portion. The recess can be sized to receive a guidewire. Alternatively, the recess can be sized to receive a portion of the medical device.

Although certain preferred examples are disclosed below, inventive subject matter extends beyond the specifically disclosed examples to other alternative examples and/or uses and to modifications and equivalents thereof. Thus, the scope of the claims that may arise herefrom is not limited by any of the particular examples described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain examples; however, the order of description should not be construed to imply that these operations are order dependent. Additionally, the structures, systems, and/or devices described herein may be embodied as integrated components or as separate components. For purposes of comparing various examples, certain aspects and advantages of these examples are described. Not necessarily all such aspects or advantages are achieved by any particular example. Thus, for example, various examples may be carried out in a manner that achieves or optimizes one advantage or group of advantages as taught herein without necessarily achieving other aspects or advantages as may also be taught or suggested herein.

Certain standard anatomical terms of location are used herein to refer to the anatomy of animals, and namely humans, with respect to the preferred examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, it is understood that these terms are used herein for ease of description to describe the positional relationship between element(s)/structures(s), as illustrated in the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of the subject patient or element/structure, and vice-versa.

Transcatheter delivery of medical devices in typical minimally invasive procedures can result in damage to medical devices. Medical devices can comprise one or more portions that do not have sufficient mechanical strength to withstand force applied the medical while advancing the medical device through anatomical pathways. The medical devices can be damaged from axial compression force exerted thereupon to advance the medical devices in the anatomical pathways. Some medical devices may comprise delicate portions that cannot withstand compressive forces used in transcatheter delivery processes. Susceptible portions of the medical device may deform under pressure applied during its transcatheter delivery. Medical devices may comprise fragile portions that can be damaged due to contact with blunt edges in delivery catheters resulting from bending of the delivery catheters while positioned in tortuous anatomical pathways.

Systems, devices, and methods described herein relate to tapered access sheaths configured to receive at least a portion of a medical device for transcatheter delivery of the medical device. The tapered access sheaths can provide mechanical strength to prevent or reduce damage to the medical devices, while facilitating navigation through anatomical pathways, including tortuous anatomical pathways. The tapered access sheath can comprise an access sheath shaft with a first delivery lumen extending from a proximal end of the access sheath shaft to a side opening on a distal portion of the access sheath shaft. A recess can be in fluid communication with and extend distally from the first delivery lumen. The recess can extend from a distal end of the first delivery lumen to a distal end of the access sheath shaft. For example, the recess can extend from the side opening to the distal end of the access sheath shaft. In some instances, a tapered distal end portion can be disposed distally of the side opening. For example, the recess can extend through a longitudinal dimension of the tapered distal end portion to the distal end of the access sheath shaft. A medical device can be slidably received within the first delivery lumen of the access sheath shaft. The medical device can be advanced along at least a portion of the first delivery catheter and out of the side opening.

In some instances, the recess can comprise a width configured to slidably receive at least a portion of a guidewire. No portion of the medical device and the delivery catheter carrying the medical device can be accommodated by the recess, such that the medical device is configured to remain disposed through the side opening after a portion of the medical device is advanced out of the first delivery lumen through the side opening. In some alternative instances, the recess can comprise a width configured to accommodate a proximal portion of the medical device. In some instances, the proximal portion of the medical device can have a smaller diameter than a distal portion of the medical device. The medical device can be advanced out of the first delivery lumen through the side opening until the proximal portion of the medical device can be received in the recess. The proximal portion of the medical device can slide into the recess. While the medical device is deployed from the access sheath shaft, at least a portion of the distal portion of the medical device can be configured to be disposed distally of a distal end of the access sheath shaft and extend along a path coaxial with a longitudinal axis of the recess.

Tapered access sheaths as described herein can provide protection for medical devices during transcatheter delivery. The tapered distal end portions of the tapered access sheaths can facilitate navigation through tortuous anatomical pathways. Medical devices can be received within the tapered access sheaths while being advanced to a target location, the tapered access sheaths thereby protecting the medical devices during the delivery process. The tapered access sheaths can provide column strength and/or reduce or eliminate blunt edges in delivery catheters, such as by preventing or reducing sharp bends in delivery catheters. The tapered access sheaths can provide mechanical strength to prevent or reduce deformation of the medical devices due to compressive force applied during delivery. Tapered access sheaths can provide protection for any susceptible distal portions of the medical devices that are distally disposed from a distal end of delivery catheters that would otherwise be exposed.

In some instances, the medical devices can be delivered to the heart. The medical device can include, for example, a mechanical circulatory support (MCS) device. For example, a mechanical circulatory support (MCS) device can be preloaded within a tapered access sheath while the tapered access sheath is advanced to a desired position within an anatomical pathway. The tapered access sheath can protect the mechanical circulatory support (MCS), including any exposed distal portions of the mechanical circulatory support (MCS) at a distal end of a delivery catheter.

As used herein, a “delivery lumen” can refer to any number of lumens, channels, passages, and/or conduits through which a medical instrument and/or medical device can be advanced, including for example a working channel. For example, a “delivery lumen” as used herein can refer to a working channel through which a medical instrument and/or medical device can be advanced for positioning the medical instrument and/or medical device at a target site within a patient.

Although the medical access sheaths and/or systems described herein are described as being inserted into the femoral artery for delivery of medical devices to the heart, it will be understood that the medical access sheaths and/or systems can be inserted into any number of other vessels and/or lumens to facilitate delivery of medical devices to any number of other organs, chambers, vessels and/or lumens. The medical access sheaths and/or systems can be used in any number of different types of minimally invasive transcatheter procedures. It will be understood that one or more components of the medical access sheaths and/or systems can undergo various processes in preparation for their use in the procedures, including for example sterilization processes. The medical access sheaths and/or systems can be sterilized medical access systems. For example, tapered access sheaths and/or introducer sheaths as described herein can be sterilized tapered access sheaths and/or sterilized introducer sheaths. Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The term “associated with” is used herein according to its broad and ordinary meaning. For example, where a first feature, element, component, device, or member is described as being “associated with” a second feature, element, component, device, or member, such description should be understood as indicating that the first feature, element, component, device, or member is physically coupled, attached, or connected to, integrated with, embedded at least partially within, or otherwise physically related to the second feature, element, component, device, or member, whether directly or indirectly.

Methods and structures disclosed herein for treating a patient also encompass analogous methods and structures performed on or placed on a simulated patient, which is useful, for example, for training; for demonstration; for procedure and/or device development; and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof; synthetic; or any combination of natural and synthetic. Virtual elements can be entirely in silica, or overlaid on one or more of the physical components. Virtual elements can be presented on any combination of screens, headsets, holographically, projected, loud speakers, headphones, pressure transducers, temperature transducers, or using any combination of suitable technologies.

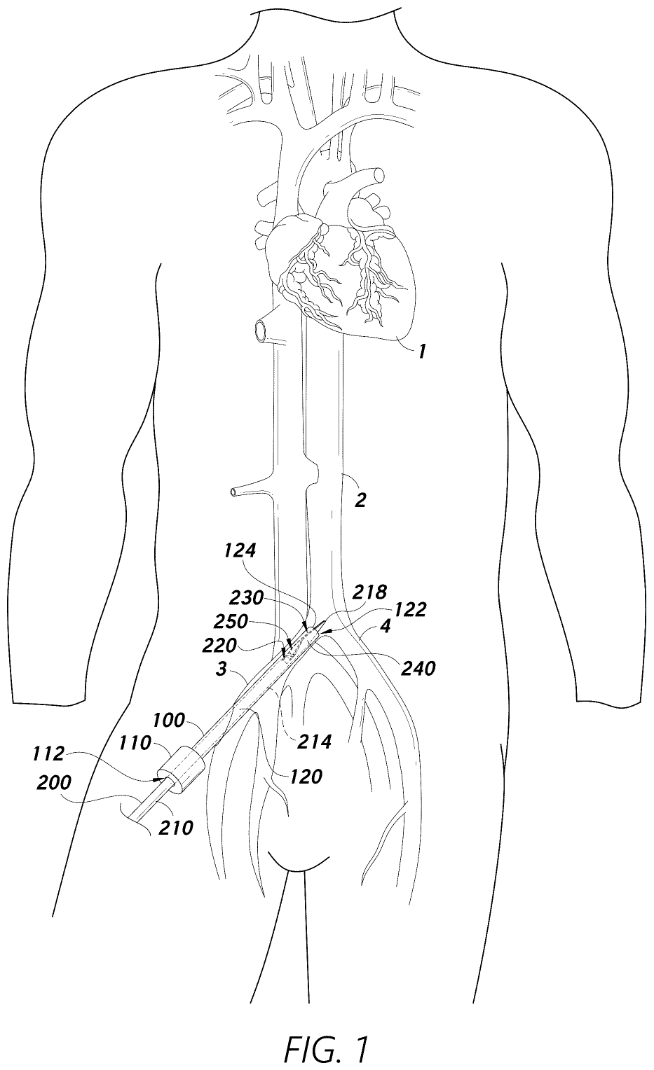

FIG. 1 shows an example of a tapered access sheath 200 comprising a portion inserted through an introducer sheath 100 comprising an introducer sheath shaft 120 positioned into a femoral artery 3 through an access opening formed thereon. The introducer sheath 100 can comprise the introducer sheath shaft 120 extending distally from an introducer hub 110. The introducer sheath shaft 120 can comprise a sheath shaft delivery lumen 122 extending therethrough and in fluid communication with a hub delivery lumen 112 extending through the introducer hub 110. The tapered access sheath 200 can comprise an access sheath shaft 210 configured to be at least partially positioned through the introducer sheath 100. For example, access sheath shaft 210 can be slidably received through the hub delivery lumen 112 and the sheath shaft delivery lumen 122 such that a portion of the access sheath shaft 210 can be disposed distally of a distal end 124 of the introducer sheath shaft 120. In some instances, the tapered access sheath 200 can be preloaded through the introducer sheath 100 while the introducer sheath 100 is positioned to a target location. The access sheath shaft 210 can comprise a tapered distal end portion 240. In some instances, the tapered access sheath 200 can be preloaded within the introducer sheath 100 while the introducer sheath 100 is navigated through an anatomical pathway such that at least a portion of the access sheath shaft 210 is slidably received through the hub delivery lumen 112 and the sheath shaft delivery lumen 122, and at least a portion of the tapered distal end portion 240 is disposed distally of the distal end 124 of the introducer sheath shaft 120.

The access sheath shaft 210 can comprise a first delivery lumen 220 extending from a proximal end of the access sheath shaft 210 to a side opening 250 on a distal portion 214 of the access sheath shaft 210. A recess 230 can be in fluid communication with the first delivery lumen 220. For example, the recess 230 can extend distally from the first delivery lumen 220 to a distal end 218 of the sheath shaft 210. The tapered distal end portion 240 can be disposed distally of the side opening 250. For example, the recess 230 can extend through a longitudinal dimension of the tapered distal end portion 240 to the distal end 218 of the access sheath shaft 210.

A medical device can be slidably received by the tapered access sheath 200. In some instances, at least a portion of the medical device can be preloaded within the first delivery lumen 220 of the access sheath shaft 210 while the access sheath shaft 210 is preloaded through the sheath shaft delivery lumen 122. For example, the introducer sheath shaft 120 carrying the access sheath shaft 210 and the medical device can be advanced along a guidewire to a target location along the anatomical pathway. In some instances, the introducer sheath shaft 120 can be deployed into the right femoral artery 3, for example remaining within the right femoral artery 3. The recess 230 can be configured to receive the guidewire. For example, while the medical device is preloaded within the tapered access sheath 200, a portion of the guidewire can be received by the medical device. A portion of the guidewire can be received by the first delivery lumen 220 distal of the medical device and another portion of the guidewire can be received by the recess 230. The medical device and guidewire are not shown in FIG. 1 for simplicity.

As described in further detail herein, the tapered access sheath 200 can be translated distally relative to the introducer sheath 100 to deploy the tapered access sheath 200 from the introducer sheath 100. In some instances, at least a portion of the tapered access sheath 200 can be deployed into the aorta 2. In some instances, the distal portion 214 of the tapered access sheath 200 can be deployed into the aorta 2. After the tapered access sheath 200 is deployed from the introducer sheath 100, the medical device can be deployed from the tapered access sheath 200. For example, at least a portion of the medical device can be advanced through the side opening 250. In some instances, at least a portion of the medical device can be advanced out of the side opening 250 disposed in the aorta 2. In some instances, the target location can be a target heart location. For example, at least a portion of the medical device can be advanced through the aorta 2 into the heart 1. It will be understood that the delivery of the medical device to the heart 1 through the right femoral artery 3 and aorta 2 is an illustrative purposes only. It will be understood that the location of the access opening is shown for illustrative purposes only. For example, the access opening can be made on any number of other vessels and/or lumens of the patient, including for example, the left femoral artery 4. The tapered access sheath 200 can be inserted into any number of other vessels and/or lumens for minimally invasive transcatheter delivery of a medical device to another target location via other delivery pathways. In some instances, tapered access sheaths can be inserted into another arterial access opening. In some instances, a venous access approach can be used. For example, the tapered access sheaths can be inserted into a femoral vein, internal jugular vein, and/or subclavian vein.

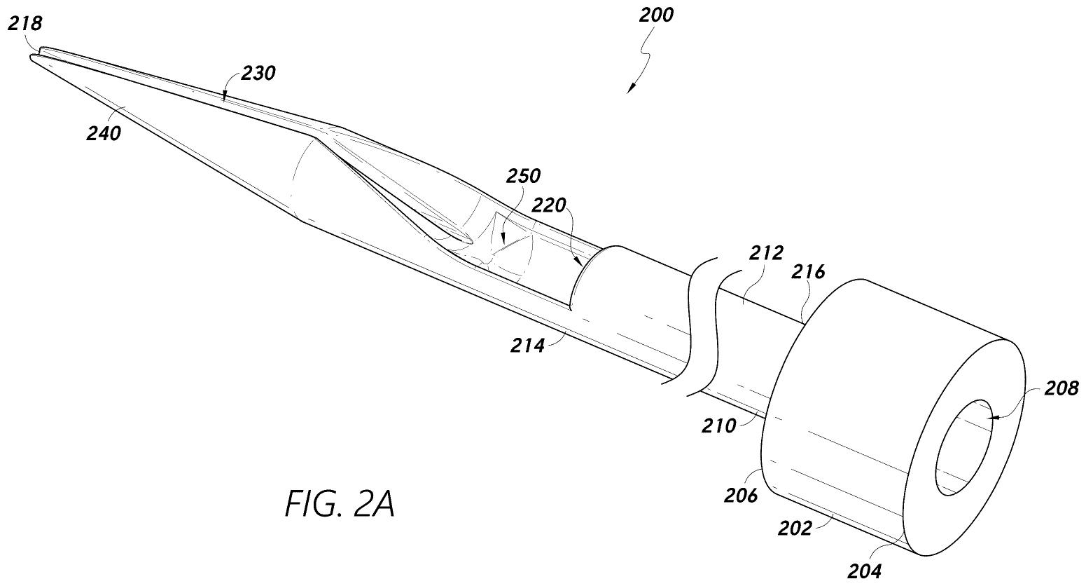

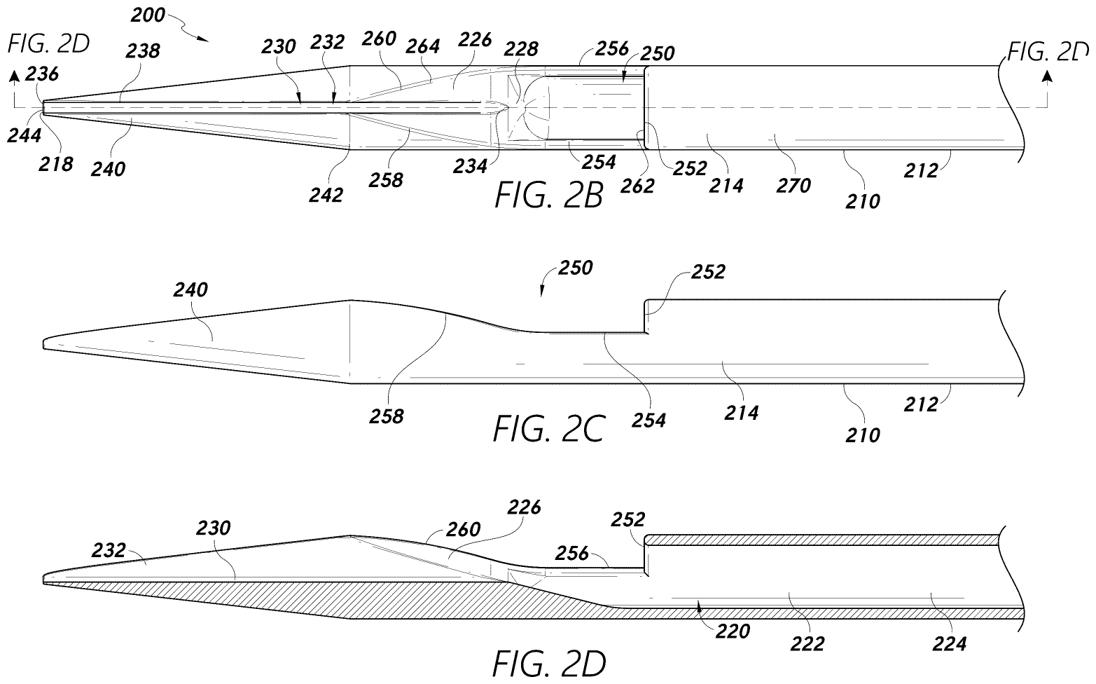

FIGS. 2A through 2D show various views of the tapered medical access sheath 200 described with reference to FIG. 1. FIG. 2A is a perspective view, FIG. 2B is a top-down view, FIG. 2C is a side view, and FIG. 2D is a longitudinal cross-sectional view of the tapered access sheath 200. The tapered access sheath 200 can comprise an access sheath hub 202 and an access sheath shaft 210 extending distally from the access sheath hub 202. The access sheath shaft 210 can be associated with a distal end 206 of the sheath hub 202. For example, a proximal portion 212 of the access sheath shaft 210 can be coupled to the distal end 206 of the sheath hub 202. The sheath hub 202 can comprise a hub delivery lumen 208 extending therethrough, from a proximal end 204 to the distal end 206. The first delivery lumen 220 of the access sheath shaft 210 can extend proximally from a side opening 250 through portions of the access sheath shaft 210 proximal of the side opening 250. For example, the first delivery lumen 220 of the access sheath shaft 210 can extend from a proximal end 216 of the access sheath shaft 210 to a side opening 250 on a distal portion 214 of the access sheath shaft 210. The first delivery lumen 220 can be sized to slidably receive a medical device or a delivery catheter carrying the medical device. In some instances, the medical device can comprise portions having different widths. In some instances, a distal portion of the medical device can have a diameter larger than a proximal portion of the medical device. The diameter can be a dimension perpendicular or substantially perpendicular to a longitudinal axis of the medical device. The first delivery lumen 220 can have a diameter wide enough to allow the distal portion of the medical device to pass through.

An inner lumen wall 222 can define the first delivery lumen 220. The inner lumen wall 222 adjacent to the side opening 250 can comprise a ramp 226. The ramp 226 can comprise a slope and/or curvature extending from a straight portion 224 of the inner lumen wall parallel or substantially parallel to the longitudinal axis of the access sheath shaft 210, to an edge of the side opening 250. The ramp 226 can be configured to guide an exit trajectory of respective portions of the medical device as the medical device is advanced through the side opening 250. For example, the ramp 226 can be configured to guide an exit trajectory of a distal portion of the medical device through the side opening 250.

A size and/or shape of the side opening 250 can be configured to facilitate exit of the medical device therethrough as guided by the ramp 226. In some instances, the side opening 250 can be defined by a proximal edge 252, a first and second lateral edge 254, 256, and a first and second distal edge 258, 260. In some instances, a sidewall portion 270 of the access sheath shaft 210 defining a proximal portion 262 of the side opening 250 can extend around a portion of a circumference of the distal portion 214 of the access sheath shaft 210. For example, the proximal edge 252 of the side opening 250 can extend along the portion of the circumference of the distal portion 214. In some instances, the proximal edge 252 can extend around about a half of the circumference of the distal portion 214. The proximal edge 252 can comprise a straight or substantially straight portion extending around a portion of the circumference of the distal portion 214, including about a half of the circumference of the distal portion 214.

The first and second lateral edges 254, 256 can extend from respective ends of the proximal edge 252. In some instances, the lateral edges 254, 256 can be parallel or substantially parallel to the longitudinal axis of the access sheath shaft 210. In some instances, the lateral edges 254, 256 can each comprise a straight or substantially straight portion. In some instances, the lateral edges 254, 256 can each be linear or substantially linear edges. For example, the first and second lateral edges 254, 256 can be perpendicular or substantially perpendicular to the proximal edge 252.

A distal portion 264 of the side opening 250 can comprise the first and second distal edges 258, 260. The first distal edge 258 can extend from the first lateral edge 254 and the second distal edge 260 can extend from the second lateral edge 256. In some instances, the first and second distal edges 258, 260 can extend from the first and second lateral edges 254, 256 toward one another, for example being angled, including at an acute angle, relative to one another. In some instances, the distal portion 264 of the side opening 250 can comprise the acute angle. In some instances, the ramp 226 can define the distal portion 264 of the side opening 250. For example, a sidewall portion 270 of the access sheath shaft 210 defining a distal portion 264 of the side opening 250 can extend along at least a portion of the ramp 226. For example, the sidewall portion 270 defining the distal portion 264 of the side opening 250 can extend along respective edges of the ramp 226.

The tapered distal end portion 240 can be disposed distally of the side opening 250. The tapered distal end portion 240 can have a taper extending from a proximal end 242 to a distal end 244. For example, a diameter of the tapered distal end portion 240 can decrease along a direction from the proximal end 242 to the distal end 244 of the tapered distal end portion 240. The diameter can be a dimension perpendicular or substantially perpendicular to the longitudinal axis of the access sheath shaft 210. The distal end 244 of the tapered distal end portion 240 can be a distal end 218 of the access sheath shaft 210. In some instances, at least a portion of the tapered distal end portion 240 can assume a truncated conical shape. For example, the distal end 244 of the tapered distal end portion 240 can be a smaller of two ends of the truncated conical shape. The proximal end 242 of the tapered distal end portion 240 can be the larger of the two ends of the truncated conical shape. The tapered distal end portion 240 can facilitate navigation of the tapered access sheath 200 through tortuous anatomical pathways. As described in further detail herein, the medical device can be slidably received by the first delivery lumen 220, including being preloaded within the first delivery lumen 220, such that the tapered access sheath 200 can protect the medical device while the medical device is advanced through the anatomical pathways. In some instances, the tapered access sheath 200 can provide mechanical strength along a longitudinal dimension for the medical device while the medical device is advanced to a target location. The tapered distal end portion 240 can facilitate positioning of the medical device to a desired location, while the medical device is received by and protected by the tapered access sheath 200.

The recess 230 can extend along a longitudinal dimension of the tapered distal end portion 240. In some instances, the recess 230 can comprise at least a portion extending from the proximal end 242 of the tapered distal end portion 240 to the distal end 244 of the tapered distal end portion 240. The recess 230 can be in fluid communication with the first delivery lumen 220. For example, the recess 230 can extend from a distal end 228 of the first delivery lumen 220 to the distal end 218 of the access sheath shaft 210. The recess 230 can extend from the side opening 250 to the distal end 218 of the access sheath shaft 210. In some instances, the tapered distal end portion 240 can be distal of and adjacent to the side opening 250. For example, the proximal end 242 of the tapered distal end portion 240 can be adjacent to a distal edge of the side opening 250, for example the first and/or second distal edge 258, 260 of the side opening 250. In some instances, recess 230 can extend from the ramp 226. For example, a proximal end 234 of the recess 230 can be at the ramp 226. The proximal end 234 of the recess 230 can extend along at least a portion of the ramp. The recess 230 can extend from the ramp 226 to a distal slit 236 on a distal end 218 of the access sheath shaft 210. In some instances, the proximal end 234 of the recess 230 can extend along the entire or substantially entire ramp 226.

An orientation of the side opening 250 can the same as or similar to that of the recess 230. For example, the opening 232 extending along the access sheath shaft 210 defined by the recess sidewall 238 can extend along a portion of the circumference that overlaps with that of the side opening 250. In some instances, the side opening 250 can extend around a first partial circumferential portion of the access sheath shaft 210. The opening 232 along the sheath shaft 210 defined by the recess 230 can extend around a second partial circumferential portion of the access sheath shaft 210. The first and second partial circumferential portions can each comprise at least a portion that overlaps with one another. In some instances, a longitudinal axis of the recess 230 can bisect the side opening 250.

In some instances, a longitudinal axis of the first delivery lumen 220 can be parallel or substantially parallel to that of the recess 230. In some instances, the first delivery lumen 220 can be coaxial with the recess 230. In some instances, the first delivery lumen 220 can be coaxial with a longitudinal axis of the access sheath shaft 210. In some instances, the recess 230 can be coaxial with a longitudinal axis of the access sheath shaft 210. In some instances, the longitudinal axis of the first delivery lumen 220 can be parallel or substantially parallel to the longitudinal axis of the access sheath shaft 210. In some instances, the longitudinal axis of the recess 230 can be parallel or substantially parallel to the longitudinal axis of the access sheath shaft 210.

The recess 230 can have a width smaller than that of the first delivery lumen 220. The width can be a dimension perpendicular or substantially perpendicular to the longitudinal axis of the access sheath shaft 210. For example, the recess 230 can have a width smaller than a diameter of the first delivery lumen 220. The recess 230 can comprise a width configured to slidably receive at least a portion of a guidewire, while not being wide enough to receive any portion of the medical device. As described in further detail herein, while the medical device is in a deployed state from the access sheath shaft, a portion of the medical device is configured to remain disposed through the side opening 250. No portion of the medical device advanced through the side opening 250 and disposed externally of the access sheath 200 can be received within the recess 230.

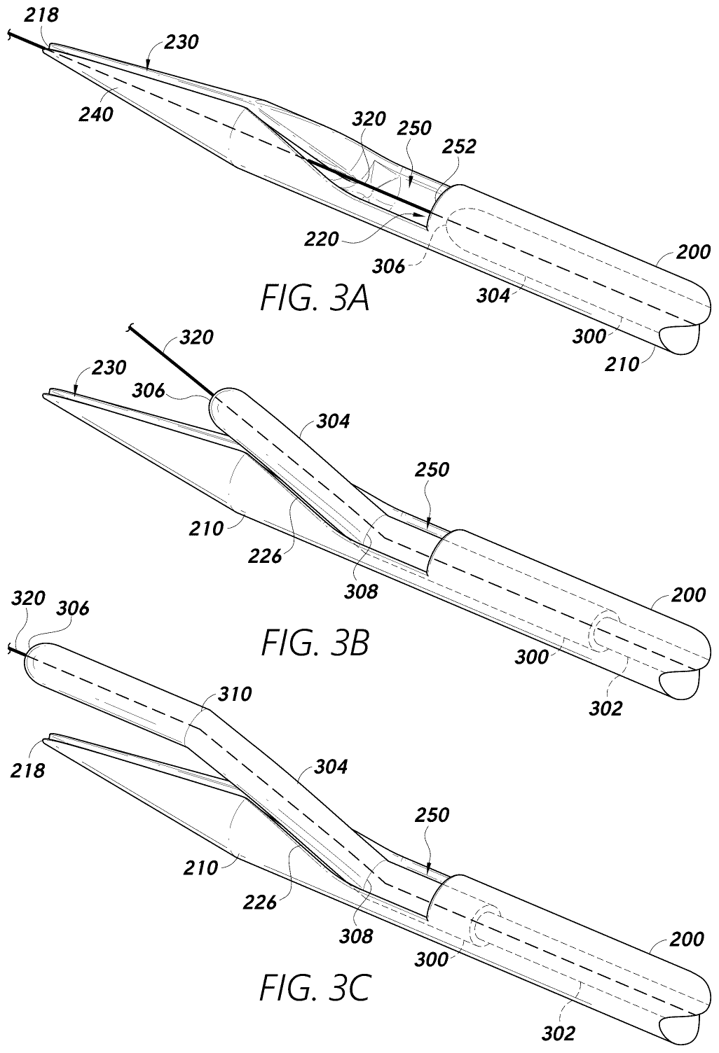

FIGS. 3A, 3B and 3C are longitudinal cross-sectional views showing delivery of a medical device 300 through the tapered access sheath 200 described with reference to FIGS. 1 and 2A through 2D. It will be understood that although the medical device 300 is described as being advanced relative to the tapered access sheath 200, the delivery process can comprise advancing a delivery catheter carrying the medical device 300 therein. Referring to FIG. 3A, in some instances, the medical device 300 can be preloaded within the tapered access sheath 200. In some instances, a distal portion 304 of the medical device 300 can be disposed within the first delivery lumen 220 extending through the distal portion 214 of the access sheath shaft 210. For example, a distal end 306 of the medical device 300 can be within the first delivery lumen 220 at a position at or proximal to the proximal edge 252 defining the side opening 250. The medical device 300 and the tapered access sheath 200 can be advanced together along a guidewire 320 to a target location. A portion of the guidewire 320 can be slidably received within a guidewire lumen of the medical device 300. A portion of the guidewire 320 extending distally from the distal end 306 of the medical device 300 can be slidably disposed in the first delivery lumen 220 and the recess 230. Alternatively, the medical device 300 can be advanced through the tapered access sheath 200 after the tapered access sheath 200 has been positioned at a desired location within an anatomical pathway. For example, after the tapered access sheath 200 has been advanced along the guidewire 320 to a target position within an anatomical pathway, at least a portion of the medical device 300 can be advanced into the first delivery lumen 220, such as advanced along the guidewire 320 into the delivery lumen 220.

After the tapered access sheath 200 carrying the medical device 300 is desirably positioned within the anatomical pathway, the medical device 300 can be advanced relative to the tapered access sheath 200 to deploy the medical device 300. In FIG. 3B, the distal portion 304 of the medical device 300 can be advanced through the side opening 250 of the tapered access sheath 200. The medical device 300 can be advanced within the first delivery lumen 220 relative to the tapered access sheath 200 to pass at least a portion of the distal portion 304 of the medical device 300 through the side opening 250. As the distal end 306 of the medical device 300 is advanced out of the side opening 250, respective portions of the medical device 300 can glide on and/or over the ramp 226 adjacent to the side opening 250 as the medical device 300 is advanced through the side opening 250. The ramp 226 can guide the exit trajectory of the respective portions of the medical device 300. The medical device 300 can form a first bend 308 as the respective portions of the medical device 300 exit through the side opening 250 and follows the trajectory guided by the ramp 226.

As the distal portion 304 of the medical device 300 exits the side opening 250, the portion of the guidewire 320 disposed distally of the medical device 300 can be lifted out of the recess 230. The guidewire 320 remains retained by the medical device 300. As the distal end 306 of the medical device is extended through the side opening 250, portions of the guidewire 320 distal of the distal end 306 of the medical device 300 can thereby be lifted from the recess 230. The medical device 300 can be continued to be advanced along the guidewire 320.

Referring to FIG. 3C, the medical device 300 can be advanced along the guidewire 320 after the distal portion 304 of the medical device 300 exits through the side opening 250. The medical device 300 can be advanced along the guidewire 320 until the distal end 306 of the medical device 300 is disposed distally of the distal end 218 of the tapered access sheath 200. In some instances, while the medical device 300 is in the deployed state from the access sheath shaft 210, at least a portion of the distal portion 304 of the medical device 300 can be configured to be disposed distally of the distal end 218 of the access sheath shaft 210. A portion of the medical device 300 disposed externally of the tapered access sheath 200 can extend along a path offset from a longitudinal axis of the tapered access sheath 200. As described herein, the recess 230 can be sized to receive the guidewire 320. The recess 230 can have a width configured to receive the guidewire 320, while no portion of the medical device 300 can be received within the recess 230. As the medical device 300 is advanced out of the side opening 250, a respective portion of the medical device 300 can remain disposed through the side opening 250.

A pathway along which portions of the medical device 300 disposed externally of the tapered access sheath 200 is advanced can be confined by the vessel and/or lumen within which the medical device 300 is deployed. For example, respective wall portions of the vessel and/or lumen within which the medical device 300 is deployed can cause the medical device 300 to bend. As the medical device 300 is further advanced out of the side opening 250 within the anatomical pathway, a portion of the medical device 300 can form a second bend 310 as the medical device 300 contacts a wall portion of the vessel and/or lumen. For example, the medical device 300 can form the first bend 308 as portions of the medical device 300 is advanced onto and/or over the ramp 226 adjacent to the side opening 250 and the second bend 310 as the medical device 300 contacts the wall portion of the vessel and/or lumen. A pathway for portions of the medical device 300 distal of the first bend 308 can be guided by the wall portion of the vessel and/or lumen. For example, portions of the medical device 300 disposed externally of the tapered access sheath 200 and/or distal of the second bend 310 can be advanced along a path outside of the tapered access sheath 200 that is offset from the longitudinal axis of the tapered access sheath 200. In some instances, the pathway can be offset and parallel or substantially parallel to the longitudinal axis of the tapered access sheath 200 and/or of the access sheath shaft 210. In some instances, the pathway can be offset and parallel or substantially parallel to the longitudinal axis of the first delivery lumen 220. In some instances, the pathway can be offset and parallel or substantially parallel to the longitudinal axis of the recess 230.

In some instances, the medical device 300 can continue to be advanced out of the tapered access sheath 200 through the side opening 250 until a portion of the proximal portion 302 is disposed through the side opening 250. After the medical device 300 is deployed to the target location, the tapered access sheath 200 can be retracted along the guidewire 320.

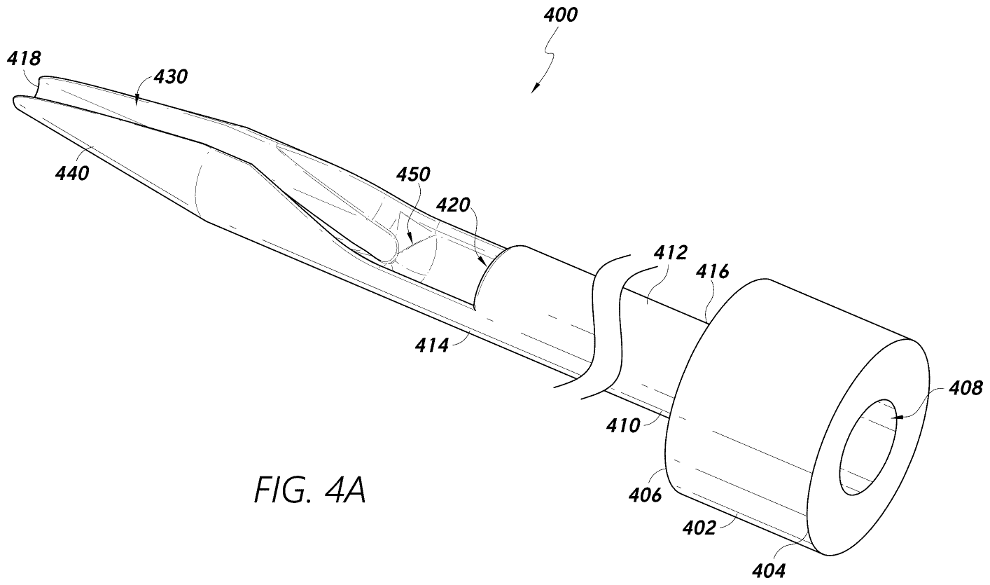

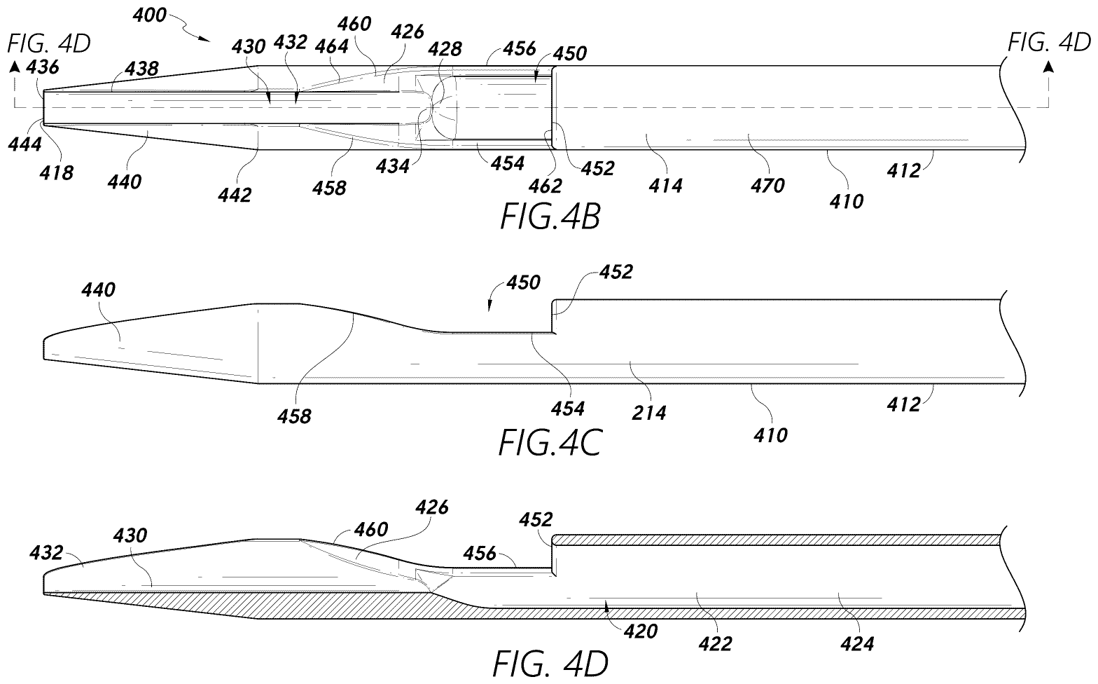

FIG. 4A is a perspective view, FIG. 4B is a top-down view, FIG. 4C is a side view, and FIG. 4D is a longitudinal cross-sectional view of an example of a tapered access sheath 400 comprising a recess 430, the tapered access sheath 400 being configured to allow a proximal portion of a medical device to be slidably disposed therethrough. The tapered access sheath 400 can comprise an access sheath shaft 410. A first delivery lumen 420 can extend from a proximal end 416 of the access sheath shaft 410 to a side opening 450 on a distal portion 414 of the access sheath shaft 410. The first delivery lumen 420 of the access sheath shaft 410 can extend proximally from the side opening 450 through portions of the access sheath shaft 410 proximal of the side opening 450. The recess 430 can be in fluid communication with the first delivery lumen 420. The recess 430 can extend from the distal end 428 of the first delivery lumen 420 to a distal end 418 of the access sheath shaft 410. The tapered access sheath 400 can be configured to receive a medical device having portions of different lateral cross-sectional sizes. In some instances, a distal portion of the medical device can have a diameter larger than more proximal portions of the medical device. The diameter can be perpendicular or substantially perpendicular to a longitudinal axis of the medical device. For example, a proximal portion of the medical device can have a diameter smaller than that of the distal portion. The first delivery lumen 420 can be sized to slidably receive the medical device or a delivery catheter carrying the medical device. The first delivery lumen 420 can have a width, such as a diameter, wide enough to allow the distal portion of the medical device to pass through. The recess 430 can have a width wide enough to accommodate the proximal portion of the medical device.

An inner lumen wall 422 defining a portion of the first delivery lumen 420 adjacent to the side opening 450 can comprise a ramp 426. The ramp 426 can extend from a straight portion 424 of the inner lumen wall 422 to a distal portion 464, including a first and second distal edge 458, 460, of the side opening 450. For example, the ramp 426 can comprise a slope and/or curvature extending from the straight portion 424 of the inner lumen wall 422. The straight portion 424 of the inner lumen wall 422 can define portions of the delivery lumen 420 which extend along a path parallel or substantially parallel to a longitudinal axis of the access sheath shaft 410.

A sidewall 470 of the access sheath shaft 410 can comprise a proximal edge 452, a first and second lateral edge 454, 456, and a first and second distal edge 458, 460 defining at least a portion of the side opening 450. In some instances, a proximal portion 462, such as the proximal edge 452, of the side opening 450 can extend along a portion of a circumference of the distal portion 414 of the access sheath shaft 410. In some instances, the proximal edge 452 can extend around about a half of the circumference of the distal portion 414. The proximal edge 452 can comprise a straight or substantially straight portion extending around a portion of the circumference of the distal portion 414, including about a half of the circumference of the distal portion 414. The first and second lateral edges 454, 456 can extend from respective ends of the proximal edge 452. In some instances, the lateral edges 454, 456 can be parallel or substantially parallel to the longitudinal axis of the access sheath shaft 410. In some instances, the lateral edges 454, 456 can each be linear or substantially linear edges. In some instances, the first and second lateral edges 454, 456 can be perpendicular or substantially perpendicular to the proximal edge 452.

the distal portion 464 of the side opening 450 can be defined at least in part by the first and second distal edges 458, 460. The first distal edge 458 and the second distal edge 460 can extend at an angle from the first lateral edge 454 and the second lateral edge 456, respectively. For example, the first and second distal edges 458, 460 can be oriented at an angle relative to one another. For example, the first and second distal edges 458, 460 can form an acute angle such that the distal portion 464 of the side opening 450 can comprise the acute angle. In some instances, the ramp 426 can define a portion of the side opening 450. For example, the first and second distal edges 458, 460 can extend along respective edges of the ramp 426.

The recess 430 can extend from the distal portion 464 of the side opening 450 to the distal end 418 of the access sheath shaft 410. For example, a proximal end 434 of the recess 430 can be at the distal portion 464 of the side opening 450. The proximal end 434 of the recess 430 can be at the first and/or second distal edges 458, 460. In some instances, recess 430 can extend from at least a portion of the ramp 426. For example, the proximal end 434 of the recess 430 can be at, on and/or along the ramp 426. The proximal end 434 of the recess 430 can extend along at least a portion of the ramp 426. In some instances, the proximal end 434 of the recess 430 can extend along the entire or substantially entire ramp 426. The recess 430 can extend from the ramp 426 to a distal slit 436 on the distal end 418 of the access sheath shaft 410.

A tapered distal end portion 440 can be disposed distally of the side opening 450. The tapered distal end portion 440 can assume a tapered profile extending from a proximal end 442 to a distal end 444. The distal end 444 of the tapered distal end portion 440 can be the distal end 418 of the access sheath shaft 410. For example, a width of the tapered distal end portion 440 can decrease along a direction from the proximal end 442 to the distal end 444. The diameter can be a dimension perpendicular or substantially perpendicular to the longitudinal axis of the access sheath shaft 410. In some instances, the tapered distal end portion 440 can assume a truncated conical shape, where the distal end 444 of the tapered distal end portion 440 can be a smaller of two ends of the truncated conical shape, and the proximal end 442 of the tapered distal end portion 440 can be the larger of the two ends of the truncated conical shape. The recess 430 can extend along a longitudinal dimension of the tapered distal end portion 440 from the proximal end 442 of the tapered distal end portion 440 to the distal end 444 of the tapered distal end portion 440. In some instances, the tapered distal end portion 440 can be distal of and adjacent to the side opening 450. For example, the proximal end 442 of the tapered distal end portion 440 can be adjacent to a distal edge of the side opening 450, for example the first and/or second distal edges 458, 460 of the side opening 450.

The recess 430 can have a width smaller than that of the first delivery lumen 420. For example, the recess 430 can have a width smaller than a diameter of the first delivery lumen 420. The width and/or diameter can be a dimension perpendicular or substantially perpendicular to the longitudinal axis of the access sheath shaft 410. The recess 430 can comprise a width configured to slidably receive a narrower portion of the medical device. For example, the recess 430 can be configured to accommodate a proximal portion of the medical device and at least a portion of a guidewire. As described in further detail herein, while the medical device is in a deployed state from the access sheath shaft 410, at least a portion of the proximal portion of the medical device can be configured to be received through the recess 430 such that the medical device can extend along a path coaxial with that of the recess 430 and/or first delivery lumen 420. For example, the distal portion of the medical device can be disposed distally of the distal end 418 of the tapered access sheath 400 along a path coaxial with the recess 430 and/or first delivery lumen 420. The proximal portion of the medical device can be configured to be disposed through the recess 430 and/or at least a portion of the first delivery lumen 420.

The tapered access sheath 400 can have one or more other features of the tapered access sheath 200 described with reference to FIGS. 1 and 2A through 2D. For example, the access sheath shaft 410 can extend distally from an access sheath hub 402. The access sheath shaft 410 can be associated with, such as coupled to, a distal end 406 of the sheath hub 402. A proximal portion 412 of the access sheath shaft 410 can be coupled to the distal end 406 of the sheath hub 402. The sheath hub 402 can comprise a hub delivery lumen 408 extending therethrough, from a proximal end 404 to the distal end 406. In some instances, a longitudinal axis of the first delivery lumen 420 can be parallel or substantially parallel to that of the recess 430 and/or hub delivery lumen 408. In some instances, the first delivery lumen 420 can be coaxial with the recess 430 and/or hub delivery lumen 408. In some instances, the first delivery lumen 420, recess 430 and/or hub delivery lumen 408 can be coaxial with a longitudinal axis of the access sheath shaft 410. In some instances, the longitudinal axis of the first delivery lumen 420, recess 430 and/or hub delivery lumen 408 can be parallel or substantially parallel to the longitudinal axis of the access sheath shaft 410. An orientation of the side opening 450 can the same as or similar to that of the recess 430. For example, the side opening 450 can extend around a first partial circumferential portion of the access sheath shaft 410. The opening 432 along the sheath shaft 410 defined by the recess 430 extends around a second partial circumferential portion of the access sheath shaft 410. The first and second partial circumferential portions can overlap with one another. In some instances, a longitudinal axis of the recess 430 can bisect the side opening 450. The opening 432 extending along the access sheath shaft 410 defined by the recess sidewall 438 can extend along a portion of the circumference that overlaps with that of the side opening 450.

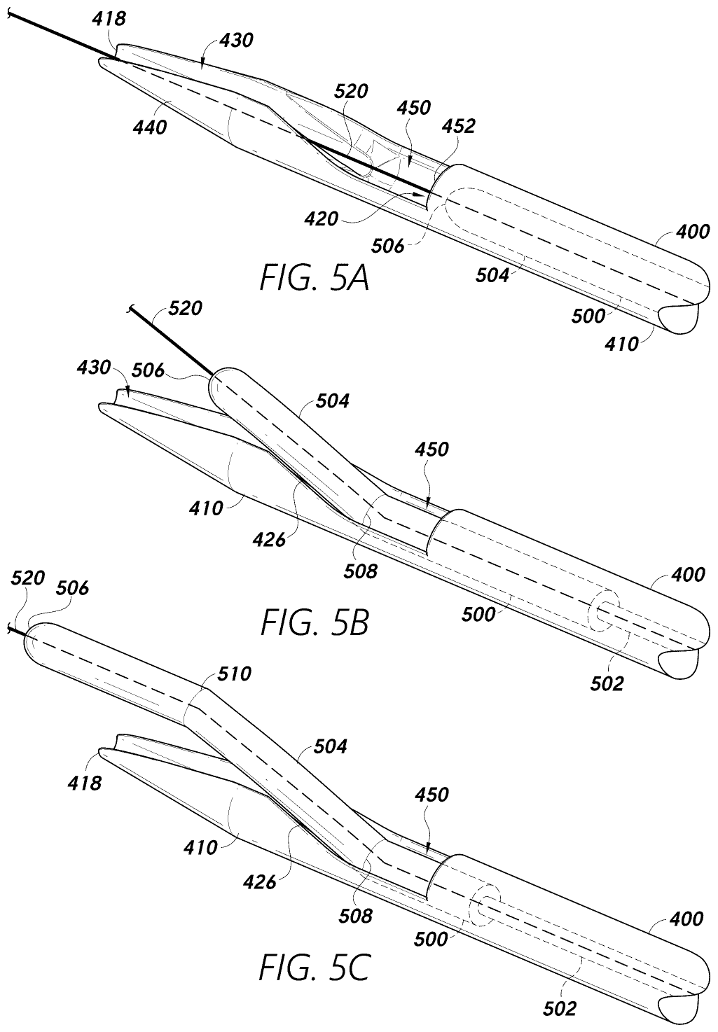

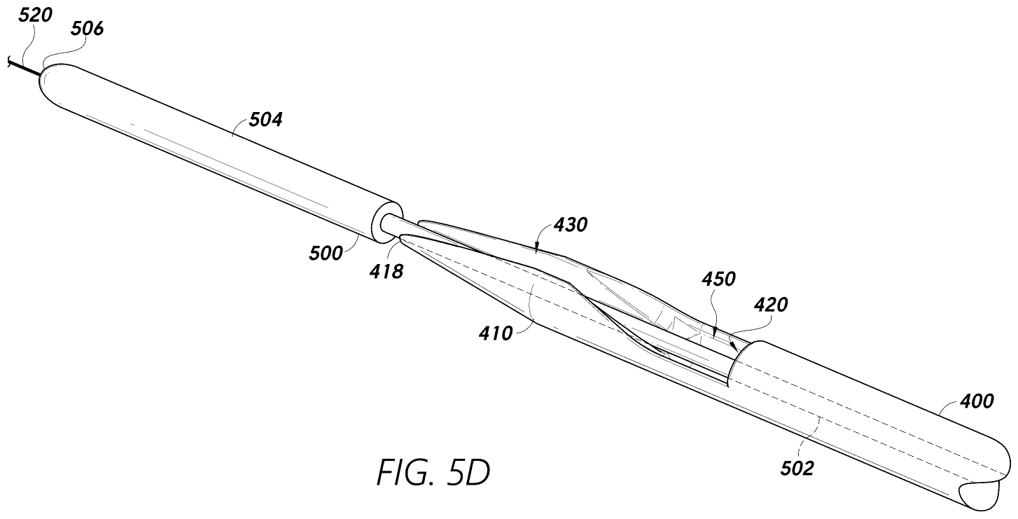

FIGS. 5A, 5B, 5C and 5D are longitudinal cross-sectional views showing delivery of a medical device 500 through the tapered access sheath 400 described with reference to FIGS. 4A through 4D. Referring to FIG. 5A, the medical device 500 is shown as being preloaded within the first delivery lumen 420 of the tapered access sheath 400. A distal portion 504 of the medical device 500 can be disposed within the first delivery lumen 420 such that a distal end 506 of the medical device 500 can be at a position at or proximal to the proximal edge 452 defining the side opening 450. The medical device 500 and the tapered access sheath 400 can be advanced together along a guidewire 520 to a target location. A portion of the guidewire 520 can be slidably received within a guidewire lumen of the medical device 500. A portion of the guidewire 520 extending distally from the distal end 506 of the medical device 500 can be slidably received within a respective portion of the first delivery lumen 420 distal of the distal end 506 of the medical device. A portion of the guidewire 520 can be slidably received in the recess 430. For example, the guidewire 520 can extend from the first delivery lumen 420 into the recess 430. Alternatively, the medical device 500 can be advanced through the tapered access sheath 400, such as along the guidewire 520, after the tapered access sheath 400 has been positioned at a desired location within an anatomical pathway.

Referring to FIG. 5B, after the tapered access sheath 400 carrying the medical device 500 is desirably positioned within the anatomical pathway, the medical device 500 can be advanced within the first delivery lumen 420 relative to the tapered access sheath 400 to pass at least a portion of the distal portion 504 of the medical device 500 through the side opening 450. As the distal end 506 of the medical device 500 is advanced out of the side opening 450, respective portions of the medical device 500 can glide on and/or over the ramp 426 adjacent to the side opening 450 as the medical device 500 is advanced through the side opening 450. The ramp 426 can guide the exit trajectory of the respective portions of the medical device 500. The medical device can form a first bend 508 as the respective portions of the medical device 500 exit through the side opening 450 and follows the trajectory guided by the ramp 426.

As the distal portion 504 of the medical device 500 exits the side opening 450, the portion of the guidewire 520 disposed distally of the medical device 500 can be lifted out of the recess 430. The guidewire 520 can remain received through the guidewire lumen of the medical device 500. Advancement of the distal end 506 of the medical device 500 out of the side opening 450 can thereby pull the guidewire 520 disposed distally of the medical device 500 out of the recess 430. The medical device 500 can be continued to be advanced along the guidewire 520.

Referring to FIG. 5C, the medical device 500 can be advanced along the guidewire 520 after the distal portion 504 of the medical device 500 exits through the side opening 450. The medical device 500 can be advanced along the guidewire 520 until the distal end 506 of the medical device 500 is disposed distally of the distal end 418 of the tapered access sheath 400. As the distal portion 504 of the medical device 500 is advanced through the side opening 450, the distal portion 504 can be disposed externally of the tapered access sheath 400 and extend along a path offset from a longitudinal axis of the tapered access sheath 400. For example, a pathway along which portions of the medical device 500 disposed externally of the tapered access sheath 400 is advanced can be confined by the vessel and/or lumen within which the medical device 500 is deployed. Respective wall portions of the vessel and/or lumen within which the medical device 500 is deployed can cause the medical device 500 to bend. The medical device 500 can form a second bend 510 as the distal portion 504 is advanced further out of the tapered access sheath 400. As described herein, the recess 430 cannot fit the distal portion 504 of the medical device. For example, the medical device 500 can form the first bend 508 as portions of the medical device 500 is advanced onto and/or over the ramp 426 adjacent to the side opening 450 and the second bend 510 as the medical device 500 contacts the wall portion of the vessel and/or lumen. Portions of the medical device 500 distal of the second bend 510 can advanced along a path outside of the tapered access sheath 400 that is offset from the longitudinal axis of the tapered access sheath 400. In some instances, the pathway can be offset and parallel or substantially parallel to the longitudinal axis of the tapered access sheath 400, the access sheath shaft 410, the first delivery lumen 420, and/or the recess 430.

As described herein, the medical device 500 can comprise a portion too large to be received within the recess 430 and a portion of the medical device 500 that can fit within the recess 430. For example, the proximal portion 502 of the medical device 500 can have a smaller diameter than that of the distal portion 504 of the medical device 500. The recess 430 can comprise a width configured to accommodate the proximal portion 502 of the medical device 500. Referring to FIG. 5D, as the medical device 500 is advanced further out of the side opening 450, at least a portion of the proximal portion 502 of the medical device 500 can be received within the recess 430. As the portion of the medical device 500 comprising a diameter too large to be received within the recess 430 is advanced distally of the distal end of the recess 430, a corresponding portion of the medical device 500 that can be received within the recess 430 can slide into the recess 430. For example, at least a portion of the proximal portion 502 can slide into the recess 430 as the portion of the medical device 500 too large to be received within the recess 430, including the distal portion 504 of the medical device 500, is advanced past the distal end 418 of the access sheath shaft 410.

In some instances, while the medical device 500 is in a deployed state from the access sheath shaft 410, at least a portion of the proximal portion 502 of the medical device 500 can be configured to be slidably disposed through the recess 430. While the medical device 500 is in the deployed state from the access sheath shaft 410, at least a portion of the distal portion 504 of the medical device 500 can be configured to be disposed distally of the distal end 418 of the access sheath shaft 410 and extend along a path coaxial with a longitudinal axis of the tapered access sheath 400 and/or access sheath shaft 410. In some instances, the distal portion 504 of the medical device 500 can be coaxial with the first delivery lumen 420. In some instances, the distal portion 504 of the medical device can be coaxial with the recess 430.

After the medical device 500 is deployed to the target location, the tapered access sheath 400 can be retracted along the guidewire 520. It will be understood that although the medical device 500 is described as being advanced relative to the tapered access sheath 400, the delivery process can comprise advancing a delivery catheter carrying the medical device 500 therein.

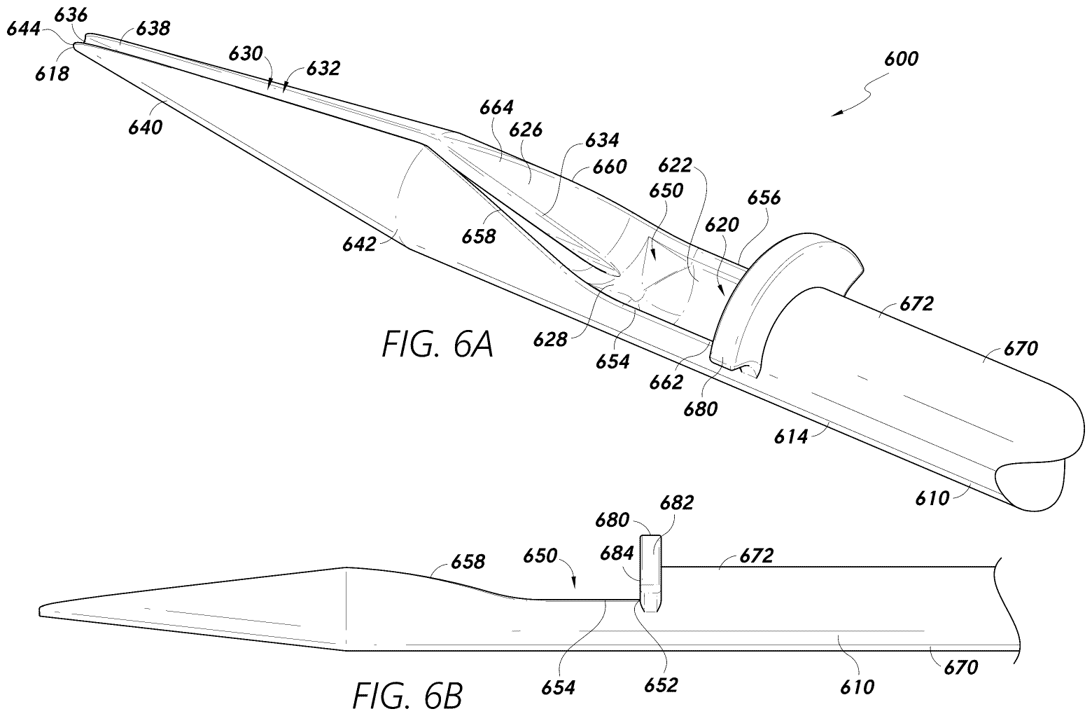

FIG. 6A is a perspective view, and FIG. 6B is a side view, of an example of a tapered access sheath 600 comprising an access sheath shaft 610 where a protrusion 680 extends along at least a portion of a sidewall portion 670 defining a side opening 650 on the access sheath shaft 610. The protrusion 680 can comprise at least a portion extending laterally of adjacent portions of the sheath shaft 610. The access sheath shaft 610 can comprise a first delivery lumen 620 extending from a proximal end to the side opening 650 on a distal portion 614 of the access sheath shaft 610. An inner lumen wall 622 defining the first delivery lumen 620 can comprise a ramp 626 adjacent to the side opening 650 extending from a straight portion 624 (not shown) of the inner lumen wall 622 that is parallel or substantially parallel to a longitudinal axis of the access sheath shaft 610 to an edge of the side opening 650. A recess 630 can extend distally from and be in fluid communication with the first delivery lumen 620. For example, the recess 630 can extend from a distal end 628 of the first delivery lumen 620 to a distal end 618 of the sheath shaft 610. A tapered distal end portion 640 can be disposed distally of the side opening 650. For example, at least a portion of the recess 630 can extend from a proximal end 642 of the tapered distal end portion 640 to a distal end 644 of the tapered distal end portion 640. The protrusion 680 can be along an exterior surface 672 of a sidewall portion 670 of the access sheath shaft 610 defining at least a portion of the side opening 650. For example, the protrusion 680 can extend along at least a portion of a proximal edge 652 of the side opening 650. In some instances, the protrusion 680 can extend along an entire or substantially entire length of the proximal edge 652. As described herein, the tapered access sheath 600 can be slidably disposed through an introducer sheath to facilitate positioning of the tapered access sheath 600. The introducer sheath can comprise a sheath shaft with an expandable distal end portion. The protrusion 680 can be configured to facilitate transformation of the expandable distal end portion from a closed state to an open state. For example, while a portion of the tapered access sheath 600 is slidably disposed through an introducer shaft delivery lumen, tapered access sheath 600 can be translated distally relative to the introducer sheath. As the tapered access sheath 600 is translated distally, the protrusion 680 can be pushed against an inner lumen wall of the expandable distal end portion so as to cause the expandable distal end portion to open and/or unfold and assume the open state.

Referring to FIG. 6A, the side opening 650 can be defined by the proximal edge 652, a first and second lateral edge 654, 656, and a first and second distal edge 658, 660 of the sidewall 670 of the access sheath shaft 610. In some instances, a sidewall portion 670 of the access sheath shaft 610 defining the proximal edge 652 of the side opening 650 can extend along a portion of a circumference of the distal portion 614 of the access sheath shaft 610. In some instances, the proximal edge 652 can extend around about a half of the circumference of the distal portion 614. The proximal edge 652 can comprise a straight or substantially straight portion extending around a portion of the circumference of the distal portion 614, including about a half of the circumference of the distal portion 614.

The first and second lateral edges 654, 656 can extend from respective ends of the proximal edge 652. In some instances, the lateral edges 654, 656 can be parallel or substantially parallel to the longitudinal axis of the access sheath shaft 610. In some instances, the lateral edges 654, 656 can each comprise a straight or substantially straight portion. In some instances, the lateral edges 654, 256 can each be linear or substantially linear edges. For example, the first and second lateral edges 654, 656 can be perpendicular or substantially perpendicular to the proximal edge 652.

A distal portion 664 of the side opening 650 can be defined at least in part by the first and second distal edges 658, 660. The first distal edge 658 and the second distal edge 660 can extend at an angle from the first lateral edge 654 and the second lateral edge 656, respectively, such that the first and second distal edges 658 can be oriented at an angle relative to one another. For example, the first and second distal edges 658, 660 can form an acute angle such that the distal portion of the side opening 650 can comprise the acute angle. In some instances, the first and second distal edges 658, 660 can extend along respective edges of the ramp 626. The protrusion 680 can be along the exterior surface 672 of the sidewall 670 defining a proximal portion 662 of the side opening 650. In some instances, the protrusion 680 can be along at least a portion of the exterior surface 672 of the sidewall 670 defining the proximal edge 652 of the side opening 650. In some instances, the protrusion 680 can be along the entire portion of the exterior surface 672 of the sidewall 670 defining the proximal edge 652 of the side opening 650. For example, the protrusion 680 can extend around half of the circumference of the distal portion 614 of the access sheath shaft 610. Alternatively, the protrusion 680 can extend along only a portion of the proximal edge 652 of the side opening 650.

Referring to FIG. 6B, the protrusion 680 can comprise a lateral surface portion 682 and a distal surface portion 684. In some instances, at least a portion of the lateral surface portion 682 and/or distal surface portion 684 can be configured to engage with the inner lumen wall of the introducer sheath to cause the expandable distal end portion of the introducer sheath to expand and/or unfold. The distal surface portion 684 of the protrusion 680 is shown as being aligned with the proximal edge 652 of the side opening 650. Alternatively, the distal surface portion 684 of the protrusion 680 can be proximal of the proximal edge 652 of the side opening 650.

The tapered access sheath 600 can comprise one or more other features of the tapered access sheath 200 described with reference to FIGS. 1 and 2A through 2D, and the tapered access sheath 400 described with reference FIGS. 4A through 4D. For example, the access sheath shaft 610 can extend distally from an access sheath hub. The access sheath shaft 610, such as a proximal portion of the access sheath shaft 610, can be coupled to a distal end of the sheath hub. The sheath hub can comprise a hub delivery lumen extending therethrough and in fluid communication with the first delivery lumen 620. In some instances, a longitudinal axis of the first delivery lumen 620 can be parallel or substantially parallel to that of the recess 630 and/or hub delivery lumen. As described herein, a proximal end 634 of the recess 630 can be at the distal end 628 of the first delivery lumen 620. The proximal end 634 of the recess 630 can extend along at least a portion of the ramp 626 to a distal slit 636 on a distal end 618 of the access sheath shaft 610. In some instances, the proximal end 634 of the recess 630 can extend along the entire or substantially entire ramp 626. In some instances, the first delivery lumen 620 can be coaxial with the recess 630 and/or hub delivery lumen. In some instances, the first delivery lumen 620, recess 630 and/or hub delivery lumen can be coaxial with a longitudinal axis of the access sheath shaft 610. In some instances, the longitudinal axis of the first delivery lumen 620, recess 630 and/or hub delivery lumen can be parallel or substantially parallel to the longitudinal axis of the access sheath shaft 610. An orientation of the side opening 650 can the same as or similar to that of the recess 630. For example, the side opening 650 can extend around a first partial circumferential portion of the access sheath shaft 610. The opening 638 along the sheath shaft 610 defined by the recess 630 extends around a second partial circumferential portion of the access sheath shaft 610. The first and second partial circumferential portions can overlap with one another. In some instances, a longitudinal axis of the recess 630 can bisect the side opening 650. The opening 638 extending along the access sheath shaft 610 defined by the recess sidewall 632 can extend along a portion of the circumference that overlaps with that of the side opening 650.

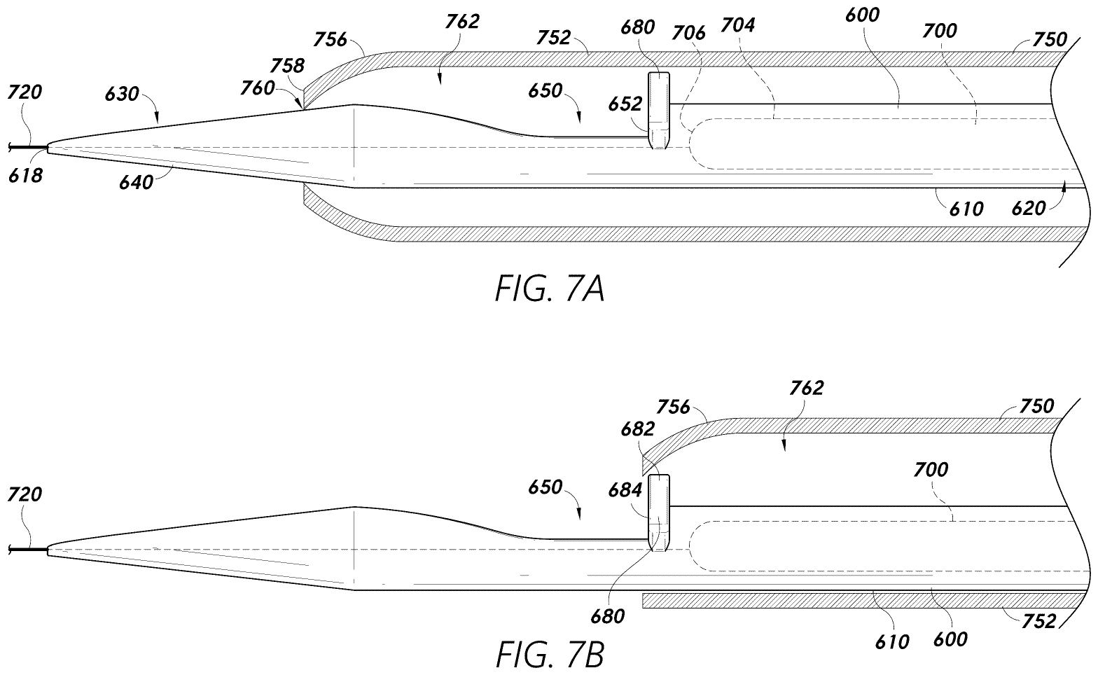

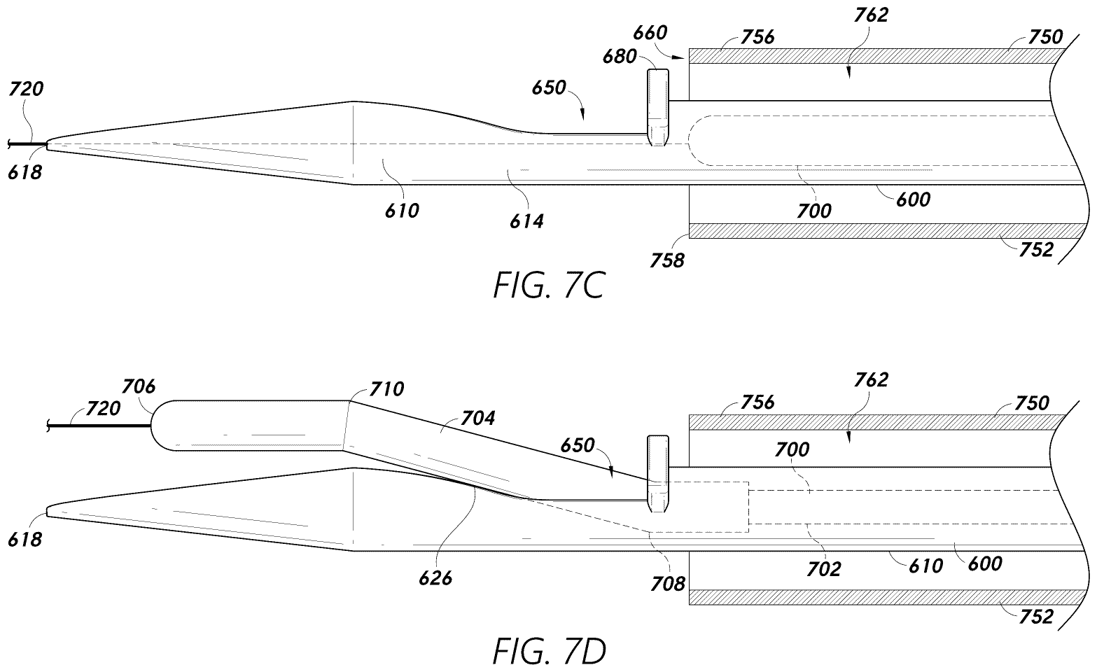

FIGS. 7A, 7B, 7C and 7D are longitudinal cross-sectional views showing delivery of a medical device 700 through the tapered access sheath 600 described with reference to FIGS. 6A and 6B. As shown in FIG. 7A, the medical device 700 can be preloaded within the first delivery lumen 620 of the access sheath shaft 610. While the medical device 700 is preloaded within the first delivery lumen 620 of the access sheath shaft 610, a distal end 706 of the medical device 700 can be within the first delivery lumen 620 at or proximal to the proximal edge 652 defining the side opening 650.

At least a portion of the access sheath shaft 610 can be received within an introducer sheath 750. The introducer sheath 750 can comprise an introducer sheath shaft 752 with an expandable distal end portion 756. The expandable distal end portion 756 is shown in a closed state in FIG. 7A. For example, at least a portion of the access sheath shaft 610 can be preloaded within a shaft delivery lumen 762 of the introducer sheath shaft 752 while the expandable distal end portion 756 is in the closed state. The medical device 700 can be preloaded within the first delivery lumen 620 of the access sheath shaft 610 while the access sheath shaft 610 is preloaded within the shaft delivery lumen 762 of the introducer sheath shaft 752. The distal end 618 of the access sheath shaft 610 can be disposed distally of a distal end 758 of the introducer sheath shaft 752. For example, at least a portion of the tapered distal end portion 640 can be disposed distally of the distal end 758 of the introducer sheath shaft 752. FIG. 7A shows a portion of the tapered distal end portion 640 disposed through a distal opening 760 at the distal end 758 of the introducer sheath shaft 752. The protrusion 680 of the tapered access sheath 600 can be within the shaft delivery lumen 762.

The introducer sheath shaft 752 carrying the access sheath shaft 610 and the medical device 700 can be advanced along a guidewire 720 to a target location within an anatomical pathway. For example, a corresponding portion of the guidewire 720 can be slidably received through the medical device 700, such as within a guidewire lumen of the medical device 700. The guidewire 720 can comprise a portion extending distally of the medical device 700 and configured to be slidably disposed through a portion of the first delivery lumen 620 distal of the preloaded medical device 700, and through the recess 630.

Referring to FIG. 7B, after the introducer sheath shaft 752 is desirably positioned, the tapered access sheath 600 can be distally translated relative to the introducer sheath 750. As described herein, the protrusion 680 can be configured to facilitate transformation of the expandable distal end portion 756 from the closed state to an open state. As the tapered access sheath 600 is translated distally, the protrusion 680 can be pushed against an inner lumen wall of the expandable distal end portion 756 so as to cause the expandable distal end portion 756 to open and/or unfold and assume the open state. One or more portions, including the lateral and/or distal surface portions 682, 684, of the protrusion 680 can contact respective portions of the inner lumen wall. In some instances, the expandable distal end portion 756 can comprise a plurality of pleats. Pushing the protrusion 680 against the inner lumen wall of the expandable distal end portion 756 can cause the plurality of pleats of the expandable distal end portion 756 to unfold.

FIG. 7C shows the tapered access sheath 600 deployed from the introducer sheath 750. The expandable distal end portion 756 can be in the open state. It will be understood that the position of the tapered access sheath 600 relative to the introducer sheath 750 is for illustrative purposes only. For example, the tapered access sheath 600 can be disposed more distally than is shown in FIG. 7C. The side opening 650 can be disposed distally of the distal end 758 of the introducer sheath shaft 752 to facilitate deployment of the medical device 700 from the tapered access sheath 600.

FIG. 7D shows deployment of the medical device 700 from the tapered access sheath 600. A portion of the distal portion 704 of the medical device 700 is disposed externally of the side opening 650 while a proximal portion 702 of the medical device 700 remains in the first delivery lumen 620. The medical device 700 can be advanced within the first delivery lumen 620 relative to the tapered access sheath 600 to pass at least a portion of the distal portion 704 of the medical device 700 through the side opening 650. As the distal end 706 of the medical device 700 is advanced out of the side opening 650, respective portions of the medical device 700 can glide on and/or over the ramp 626 adjacent to the side opening 650. The ramp 626 can guide the exit trajectory of the respective portions of the medical device 700. The medical device 700 can form a first bend 708 as the medical device exits the side opening 650 along the ramp 626.

As the distal portion 704 of the medical device 700 exits the side opening 650, the portion of the guidewire 720 disposed distally of the medical device 700 can be lifted out of the recess 630. The recess 630 can be sized such that no portion of the medical device 700 can be disposed through the recess 630. The medical device 700 can be continued to be advanced along the guidewire 720. The pathway along which portions of the medical device 700 disposed externally of the tapered access sheath 600 is advanced can be confined by the vessel and/or lumen within which the medical device 700 is deployed. The medical device 700 can form a second bend 710 as the distal portion 704 is advanced further out of the tapered access sheath 600 and contacts wall portions of the vessel and/or lumen. Portions of the medical device 700 disposed externally of the tapered access sheath 600 can follow a pathway of one or more medical devices as described herein. Alternatively, the recess 630 can be sized to receive a portion of the medical device. For example, the recess 630 can have a width configured to slidably receive a narrower portion of the medical device 700, such as being configured to accommodate a proximal portion 702 of the medical device 700.

Although delivery processes described herein comprise a medical device preloaded within a tapered access sheath, it will be understood that, alternatively, the medical device can be advanced through the tapered access sheath after the tapered access sheath has been positioned at a desired location within an anatomical pathway. For example, after the tapered access sheath has been advanced along a guidewire to a target position within an anatomical pathway, at least a portion of the medical device can be advanced into a first delivery lumen of the taper access sheath.

In some instances, a medical device delivery system can comprise a tapered access sheath as described herein. For example, the medical device delivery system can comprise a delivery catheter and a medical device comprising at least a portion configured to be received by the delivery catheter. The delivery catheter can comprise a catheter delivery lumen extending therethrough, and at least a portion of the medical device can be slidably received by the catheter delivery lumen. A tapered access sheath comprising one or more features described herein can be configured to slidably receive at least a portion of the delivery catheter and the medical device received by the delivery catheter. For example, the tapered access sheath can comprise an access sheath hub and an access sheath shaft extending distally from the access sheath hub. The access sheath shaft can comprise a first delivery lumen extending from a proximal end of the access sheath shaft to a side opening on a distal portion of the access sheath shaft. The access sheath shaft can comprise a tapered distal end portion disposed distally of the side opening. A recess can extend along a longitudinal dimension of the tapered distal end portion from a proximal end to a distal end of the tapered distal end portion. The recess can be in fluid communication with the first delivery lumen. In some instances, an orientation of the side opening can be the same as or similar to that of the recess. For example, the side opening can extend around a first partial circumferential portion of the access sheath shaft. The recess can extend around a second partial circumferential portion of the access sheath shaft. The first and second partial circumferential portions can each comprise at least a portion that overlaps with one another. In some instances, a longitudinal axis of the first delivery lumen can be parallel or substantially parallel to that of the recess. In some instances, the longitudinal axes of the first delivery lumen and of the recess can be parallel or substantially parallel to a longitudinal axis of the access sheath shaft.

The delivery catheter carrying the medical device can be preloaded within the first delivery lumen of the tapered access sheath. For example, a distal end of the delivery catheter can be configured to be received within the first delivery lumen while the delivery catheter is preloaded within the tapered access sheath. In some instances, the medical device can be preloaded within the delivery catheter. In some instances, at least a portion of a distal portion of the medical device can be disposed distally of a distal end of the delivery catheter while the medical device is preloaded within the delivery catheter. For example, a distal end of the medical device can be configured to be disposed distally of a distal end of the delivery catheter. In some instances, while the delivery catheter carrying the medical device is preloaded within the first delivery lumen, the distal end of the medical device can proximal of and adjacent to a proximal edge of the side opening of the tapered access sheath.

The recess can have a width smaller than that of the first delivery lumen. As described herein, in some instances, the recess can comprise a width configured to slidably receive at least a portion of a guidewire. No portion of the medical device and the delivery catheter carrying the medical device can be accommodated by the recess, such that a portion of the delivery catheter carrying the medical device is configured to remain disposed through the side opening after a portion of the delivery catheter carrying the medical device is advanced out of the first delivery lumen through the side opening. A portion of the delivery catheter receiving the medical device can be disposed through the side opening while the delivery catheter and the medical device are deployed from the access sheath shaft. For example, while the delivery catheter receiving the medical device is deployed from the access sheath shaft, at least a portion of a distal portion of the delivery catheter receiving the medical device can be configured to be disposed distally of a distal end of the access sheath shaft and to extend along a path offset from a longitudinal axis of the recess.

As described herein, in some alternative instances, the recess can comprise a width configured to accommodate a proximal portion of the delivery catheter receiving the medical device. In some instances, the proximal portion of the medical device and the proximal portion of the delivery catheter receiving the medical device can have a smaller diameter than a distal portion of the medical device and the delivery catheter receiving the medical device, respectively. For example, as the distal portion of the delivery catheter carrying the distal portion of the medical device is advanced out of the first delivery lumen through the side opening, a portion of the delivery catheter carrying the medical device can be disposed through the side opening. As the delivery catheter carrying the medical device is further advanced out of the first delivery lumen through the side opening, the proximal portion of the delivery catheter carrying the proximal portion of the medical device can be advanced out through the side opening. The proximal portion of the delivery catheter receiving the proximal portion of the medical device can slide into the recess. For example, while the delivery catheter receiving the medical device is deployed from the access sheath shaft, at least a portion of the proximal portion of the delivery catheter receiving the proximal portion of the medical device can be configured to be slidably disposed through the recess. While the delivery catheter receiving the medical device is deployed from the access sheath shaft, at least a portion of the distal portion of the delivery catheter receiving the distal portion of the medical device can be configured to be disposed distally of a distal end of the access sheath shaft and extend along a path coaxial with a longitudinal axis of the recess.

In some instances, a protrusion along an exterior surface of a sidewall portion of the access sheath shaft defining at least a portion of the side opening. The protrusion can be configured to be pushed against an inner lumen wall of an expandable distal end portion of an introducer sheath while the tapered access sheath is slidably disposed through an introducer sheath delivery lumen and translated distally relative to the introducer sheath. The protrusion can be along at least a portion of the sidewall portion defining a proximal edge of the side opening. For example, the protrusion can be along an entire or substantially entire proximal edge of the side opening.

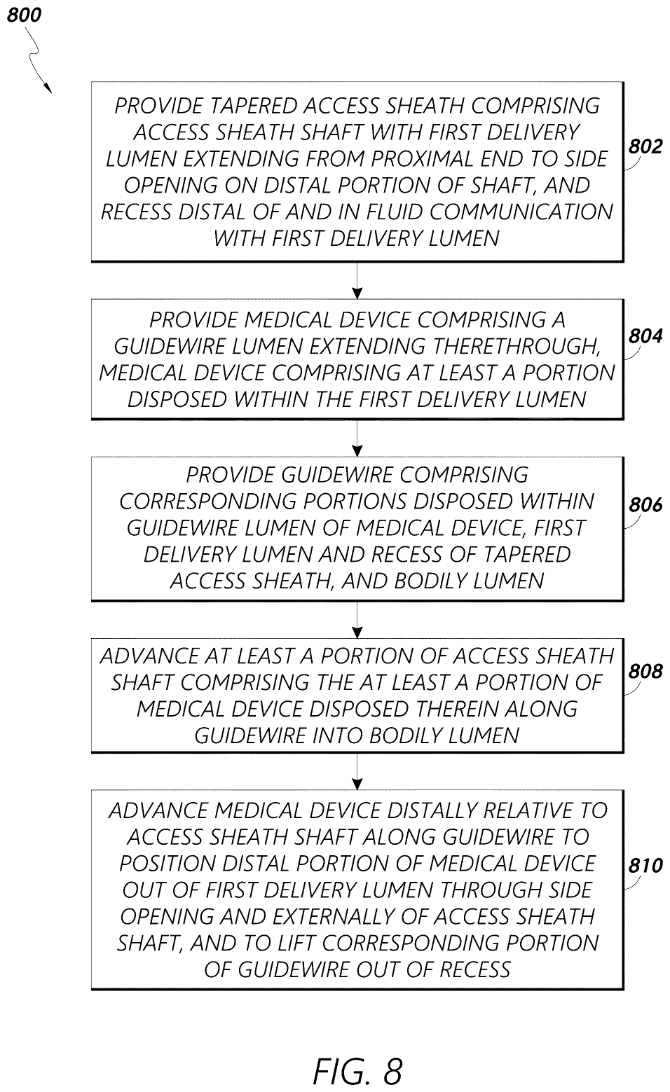

FIG. 8 is a process flow diagram of an example of a process 800 for delivering a medical device using a tapered medical access sheath as described herein. In block 802, the process involves providing a tapered access sheath. The tapered access sheath can comprise an access sheath shaft having a tapered distal end portion. The access sheath shaft can comprise a first delivery lumen extending from a proximal end of the access sheath shaft to a side opening on a distal portion of the access sheath shaft. The side opening can be proximal of the tapered distal end portion. The access sheath shaft can comprise a recess distal of and in fluid communication with the first delivery lumen. For example, the recess can extend through the tapered distal end portion and be in fluid communication with the first delivery lumen. The recess can have a width smaller than that of the first delivery lumen.

In block 804, the process can involve providing a medical device comprising a guidewire lumen extending therethrough. The medical device can comprise at least a portion disposed within the first delivery lumen. In block 806, the process can involve providing a guidewire comprising corresponding portions disposed within the guidewire lumen of the medical device, the first delivery lumen and the recess of the tapered access sheath, and a bodily lumen. In block 808, the process can involve advancing at least a portion of the access sheath shaft comprising the at least a portion of the medical device disposed therein along the guidewire into a bodily lumen.

In block 810, the process can involve advancing the medical device distally relative to the access sheath shaft along the guidewire to position a distal portion of the medical device out of the first delivery lumen through the side opening and externally of the access sheath shaft. A corresponding portion of the guidewire can be lifted out of the recess. For example, as a distal end of the medical device is advanced out of the first delivery lumen out of the side opening, portions of the guidewire extending distally from the distal end of the medical device can be lifted out of the recess.

In some instances, the recess can be sized to accommodate the guidewire. No portion of the medical device can be received by the recess. For example, advancing the medical device distally relative to the access sheath shaft along the guidewire can comprise positioning at least a portion of the distal portion of the medical device disposed externally of the first delivery lumen along an axis offset from a longitudinal axis of the recess. Positioning the at least a portion of the distal portion of the medical device disposed externally of the first delivery lumen can comprise positioning the at least a portion of the distal portion of the medical device along an axis parallel or substantially parallel to the longitudinal axis of the recess. A portion of the medical device can remain disposed through the side opening while portions of the medical device are advanced out of the first delivery lumen through the side opening.

In some instances, the recess can be sized to accommodate a portion of the medical device. For example, the medical device can comprise a proximal portion having a smaller diameter than a distal portion. The proximal portion of the medical device can be received by the recess. In some instances, advancing the medical device distally relative to the access sheath shaft along the guidewire can comprise advancing the medical device until a portion of the medical device fits within the recess. The portion of the medical device can be positioned through the recess. For example, the proximal portion of the medical device can be positioned through the recess. The distal portion of the medical device can be disposed distally of a distal end of the access sheath shaft. In some instances, disposing the distal portion of the medical device distally of the distal end of the access sheath shaft can comprise coaxially disposing the distal portion of the medical device and the recess.

In some instances, advancing at least a portion of the access sheath shaft comprising the at least a portion of the medical device disposed therein along the guidewire into a bodily lumen can comprise advancing at least a portion of the access sheath shaft comprising the at least a portion of the medical device disposed therein along the guidewire into a femoral artery. As described herein, at least a portion of the medical device can be delivered into the heart. The medical device can be advanced into the aorta from the femoral artery. In some instances, the medical device can be advanced into the heart from the aorta. In some instances, at least a portion of the medical device can be delivered into a chamber of the heart, including a left heart chamber. For example, at least a portion of the medical device can be delivered into a left ventricle of the heart.

In some instances, the medical device can comprise at least a portion configured to be received within a delivery catheter. For example, providing the medical device can comprise providing the medical device received by a catheter delivery lumen of a delivery catheter. At least a portion of the medical device can be received within the catheter delivery lumen. Advancing at least a portion of the access sheath shaft comprising the at least a portion of the medical device disposed therein along the guidewire into a bodily lumen can comprise advancing at least a portion of the access sheath shaft comprising the delivery catheter receiving the medical device disposed therein. Advancing the medical device distally relative to the access sheath shaft along the guidewire to position a distal portion of the medical device out of the first delivery lumen through the side opening can comprise advancing the delivery catheter receiving the medical device relative to the access sheath shaft along the guidewire. The delivery catheter carrying the medical device can be advanced so as to position a distal portion of the delivery catheter receiving the distal portion of the medical device out of the first delivery lumen. In some instances, the medical device can be advanced distally relative to the delivery catheter to deploy the medical device from the delivery catheter. For example, after the delivery catheter is desirably positioned, the medical device can be deployed from the delivery catheter.

In some instances, the tapered access sheath can be received by an introducer sheath. For example, the access sheath shaft can be slidably received in an introducer sheath shaft while the introducer sheath is positioned into the bodily lumen. In some instances, positioning the introducer sheath into the bodily lumen can comprise advancing the introducer sheath into a femoral artery and maintaining a distal end of the introducer sheath within the femoral artery. Advancing at least a portion of the access sheath shaft comprising the at least a portion of the medical device disposed therein along the guidewire into a bodily lumen can comprise advancing the at least a portion of the access sheath shaft and medical device through a sheath delivery lumen of the introducer sheath positioned into the bodily lumen. Advancing the at least a portion of the access sheath shaft and medical device through the introducer sheath can comprise advancing the at least a portion of the access sheath shaft and medical device from the femoral artery into an aorta. Advancing the medical device distally relative to the access sheath shaft along the guidewire to position a distal portion of the medical device out of the first delivery lumen through the side opening can comprise advancing the medical device distally relative to the access sheath shaft along the guidewire while a distal end of the access sheath shaft is within the aorta. At least a portion of the medical device can be advanced through the introducer sheath and into the heart.

In some instances, a protrusion can be on an exterior surface of a side wall portion of the access sheath shaft defining a proximal edge of the side opening. The protrusion can be used to engage with an inner lumen wall portion of the introducer sheath shaft. The introducer sheath shaft can comprise an expandable distal end portion. Advancing the at least a portion of the access sheath shaft and medical device through the introducer sheath can comprise pushing the protrusion against an inner lumen wall portion of the expandable distal end portion of the introducer sheath to expand the expandable distal end portion. For example, pushing the protrusion against the inner lumen wall can cause the expandable distal end portion to open, transforming into an open state from a closed state.

### Additional Description of Examples

Provided below is a list of examples, each of which may include aspects of any of the other examples disclosed herein. Furthermore, aspects of any example described above may be implemented in any of the numbered examples provided below.

Example 1: A tapered medical access sheath comprising an access sheath hub, and an access sheath shaft extending distally from the access sheath hub. The access sheath shaft can comprise a first delivery lumen extending from a proximal end of the access sheath shaft to a side opening on a distal portion of the access sheath shaft, a tapered distal end portion disposed distally of the side opening, and a recess extending along a longitudinal dimension of the tapered distal end portion from a proximal end to a distal end of the tapered distal end portion, the recess being in fluid communication with the first delivery lumen and having a width smaller than that of the first delivery lumen.

Example 2: The sheath of any example herein, in particular example 1, wherein a longitudinal axis of the first delivery lumen is parallel to that of the recess.

Example 3: The sheath of any example herein, in particular example 1 or 2, wherein the first delivery lumen is coaxial with the recess.

Example 4: The sheath of any example herein, in particular examples 1 to 3, wherein the first delivery lumen and the recess are coaxial with a longitudinal axis of the access sheath shaft.

Example 5: The sheath of any example herein, in particular examples 1 to 4, wherein longitudinal axes of the first delivery lumen and of the recess are parallel to a longitudinal axis of the access sheath shaft.

Example 6: The sheath of any example herein, in particular examples 1 to 5, wherein an orientation of the side opening is the same as that of the recess.

Example 7: The sheath of any example herein, in particular examples 1 to 6, wherein the side opening extends around a first partial circumferential portion of the access sheath shaft and the recess extends around a second partial circumferential portion of the access sheath shaft, the first and second partial circumferential portions each comprising at least a portion that overlaps with one another.

Example 8: The sheath of any example herein, in particular examples 1 to 7, wherein the first delivery lumen is configured to slidably receive at least a portion of a medical device, an inner lumen wall portion defining a portion of the first delivery lumen adjacent to the side opening comprises a ramp configured to guide an exit trajectory of a distal portion of the medical device through the side opening, and the recess extends from at least a portion of the ramp to a distal opening on a distal end of the access sheath shaft.

Example 9: The sheath of any example herein, in particular examples 1 to 8, wherein the recess comprises a width configured to slidably receive at least a portion of a guidewire, the first delivery lumen comprises a diameter configured to slidably receive at least a portion of a medical device, and while the medical device is in a deployed state from the access sheath shaft, a portion of the medical device is configured to remain disposed through the side opening.

Example 10: The sheath of any example herein, in particular example 9, wherein, while the medical device is in the deployed state from the access sheath shaft, at least a portion of a distal portion of the medical device is configured to be disposed distally of a distal end of the access sheath shaft and to extend along a path offset from a longitudinal axis of the recess.

Example 11: The sheath of any example herein, in particular examples 1 to 8, wherein the recess comprises a width configured to accommodate a proximal portion of a medical device, the proximal portion of the medical device having a smaller diameter than that of a distal portion of the medical device; the first delivery lumen comprises a diameter configured to accommodate the distal portion of the medical device, and while the medical device is in a deployed state from the access sheath shaft, at least a portion of the proximal portion of the medical device is configured to be slidably disposed through the recess.

Example 12: The sheath of any example herein, in particular example 11, wherein while the medical device is in the deployed state from the access sheath shaft, at least a portion of the distal portion of the medical device is configured to be disposed distally of a distal end of the access sheath shaft and extend along a path coaxial with a longitudinal axis of the recess.

Example 13: The sheath of any example herein, in particular examples 1 to 12, wherein the tapered distal end portion is distal of and adjacent to a distal edge of the side opening.

Example 14: The sheath of any example herein, in particular examples 1 to 13, wherein a sidewall portion of the access sheath shaft defining a proximal edge of the side opening extends around a portion of a circumference of the distal portion of the access sheath shaft.

Example 15: The sheath of any example herein, in particular example 14, wherein the sidewall portion of the access sheath shaft defining a proximal edge of the side opening extends around a half of the circumference of the distal portion of the access sheath shaft.

Example 16: The sheath of any example herein, in particular examples 1 to 15, further comprising a protrusion along an exterior surface of a sidewall portion of the access sheath shaft defining at least a portion of the side opening, the protrusion being configured to be pushed against an inner lumen wall of an expandable distal end portion of an introducer sheath while the tapered access sheath is slidably disposed through an introducer sheath delivery lumen and translated distally relative to the introducer sheath.

Example 17: The sheath of any example herein, in particular example 16, wherein the protrusion is along at least a portion of a sidewall portion of the access sheath shaft defining a proximal edge of the side opening.

Example 18: The sheath of any example herein, in particular example 16 or 17, wherein the protrusion is along a sidewall portion of the access sheath shaft defining a proximal edge of the side opening, the proximal edge of the side opening extending around a portion of a circumference of the access sheath shaft.

Example 19: The sheath of any example herein, in particular examples 1 to 18, wherein an inner lumen wall portion defining a portion of the first delivery lumen adjacent to the side opening comprises a ramp configured to guide an exit trajectory of a distal portion of a medical device through the side opening, and a sidewall portion of the access sheath shaft defining a distal portion of the side opening extends along at least a portion of the ramp.

Example 20: The sheath of any example herein, in particular example 19, wherein the sidewall portion defining the distal portion of the side opening extends along respective edges of the ramp.

Example 21: The sheath of any example herein, in particular examples 1 to 20, wherein the tapered access sheath is sterilized.

Example 22: A method of delivering a medical device, the method comprising providing a tapered access sheath comprising an access sheath shaft having a tapered distal end portion, and the access sheath shaft further comprising a first delivery lumen extending from a proximal end of the access sheath shaft to a side opening on a distal portion of the access sheath shaft, the side opening being proximal of the tapered distal end portion. A recess can extend through the tapered distal end portion and be in fluid communication with the first delivery lumen, the recess having a width smaller than that of the first delivery lumen. The method can include providing a medical device comprising a guidewire lumen extending therethrough, the medical device comprising at least a portion disposed within the first delivery lumen, and providing a guidewire comprising corresponding portions disposed within the guidewire lumen of the medical device, the first delivery lumen and the recess of the tapered access sheath, and a bodily lumen. The method can include advancing at least a portion of the access sheath shaft comprising the at least a portion of the medical device disposed therein along the guidewire into a bodily lumen, and advancing the medical device distally relative to the access sheath shaft along the guidewire to position a distal portion of the medical device out of the first delivery lumen through the side opening and externally of the access sheath shaft, and to lift a corresponding portion of the guidewire out of the recess.

Example 23: The method of any example herein, in particular example 22, wherein advancing the medical device distally relative to the access sheath shaft along the guidewire comprises positioning at least a portion of the distal portion of the medical device disposed externally of the first delivery lumen along an axis offset from a longitudinal axis of the recess.

Example 24: The method of any example herein, in particular example 23, wherein positioning the at least a portion of the distal portion of the medical device disposed externally of the first delivery lumen comprises positioning the at least a portion of the distal portion of the medical device along an axis parallel to the longitudinal axis of the recess.

Example 25: The method of any example herein, in particular examples 22 to 24, further comprising maintaining a portion of the medical device disposed through the side opening after advancing the portions of the medical device out of the first delivery lumen through the side opening.

Example 26: The method of any example herein, in particular examples 22 to 24, wherein advancing the medical device distally relative to the access sheath shaft along the guidewire comprises: advancing the medical device until a portion of the medical device fits within the recess; positioning the portion of the medical device through the recess; and disposing the distal portion of the medical device distally of a distal end of the access sheath shaft.

Example 27: The method of any example herein, in particular example 26, wherein disposing the distal portion of the medical device distally of the distal end of the access sheath shaft comprises coaxially disposing the distal portion of the medical device and the recess.

Example 28: The method of any example herein, in particular examples 22 to 27, wherein advancing at least a portion of the access sheath shaft comprising the at least a portion of the medical device disposed therein along the guidewire into a bodily lumen comprises advancing at least a portion of the access sheath shaft comprising the at least a portion of the medical device disposed therein along the guidewire into a femoral artery.

Example 29: The method of any example herein, in particular examples 22 to 28, further comprising positioning an introducer sheath into the bodily lumen, and wherein advancing at least a portion of the access sheath shaft comprising the at least a portion of the medical device disposed therein along the guidewire into a bodily lumen comprises advancing the at least a portion of the access sheath shaft and medical device through a sheath delivery lumen of the introducer sheath positioned into the bodily lumen.

Example 30: The method of any example herein, in particular example 29, wherein positioning the introducer sheath into the bodily lumen comprises advancing the introducer sheath into a femoral artery and maintaining a distal end of the introducer sheath within the femoral artery.

Example 31: The method of any example herein, in particular example 30, wherein advancing the at least a portion of the access sheath shaft and medical device through the introducer sheath comprises advancing the at least a portion of the access sheath shaft and medical device from the femoral artery into an aorta.

Example 32: The method of any example herein, in particular example 31, wherein advancing the medical device distally relative to the access sheath shaft along the guidewire to position a distal portion of the medical device out of the first delivery lumen through the side opening comprises advancing the medical device distally relative to the access sheath shaft along the guidewire while a distal end of the access sheath shaft is within the aorta.

Example 33: The method of any example herein, in particular examples 29 to 32, wherein advancing the at least a portion of the access sheath shaft and medical device through the introducer sheath comprises advancing the at least a portion of the medical device into a heart.

Example 34: The method of any example herein, in particular examples 29 to 33, wherein advancing the at least a portion of the access sheath shaft and medical device through the introducer sheath comprises pushing a protrusion on an exterior surface of a side wall portion of the access sheath shaft defining a proximal edge of the side opening against an inner lumen wall portion of an expandable distal end portion of the introducer sheath to expand the expandable distal end portion.

Example 35: The method of any example herein, in particular examples 22 to 34, wherein providing the medical device comprises providing the medical device received by a catheter delivery lumen of a delivery catheter, advancing at least a portion of the access sheath shaft comprising the at least a portion of the medical device disposed therein along the guidewire into a bodily lumen comprises advancing at least a portion of the access sheath shaft comprising the delivery catheter receiving the medical device disposed therein, and advancing the medical device distally relative to the access sheath shaft along the guidewire to position a distal portion of the medical device out of the first delivery lumen through the side opening comprises advancing the delivery catheter receiving the medical device relative to the access sheath shaft along the guidewire to position a distal portion of the delivery catheter receiving the distal portion of the medical device out of the first delivery lumen.

Example 36: The method of any example herein, in particular example 35, further comprising advancing the medical device distally relative to the delivery catheter to deploy the medical device from the delivery catheter.

The above method(s) can be performed on a living animal or on a simulation, such as on a cadaver, cadaver heart, anthropomorphic ghost, simulator (e.g., with body parts, heart, tissue, etc. being simulated).

Example 37: A medical device delivery system comprising a delivery catheter including a catheter delivery lumen extending therethrough, and at least a portion of a medical device slidably received by the catheter delivery lumen. The system can include a tapered access sheath configured to slidably receive at least a portion of the delivery catheter and the medical device received by the delivery catheter. The tapered access sheath can comprise an access sheath hub, and an access sheath shaft extending distally from the access sheath hub. The access sheath shaft can comprise a first delivery lumen extending from a proximal end of the access sheath shaft to a side opening on a distal portion of the access sheath shaft, a tapered distal end portion disposed distally of the side opening, and a recess extending along a longitudinal dimension of the tapered distal end portion from a proximal end to a distal end of the tapered distal end portion, the recess being in fluid communication with the first delivery lumen and having a width smaller than that of the first delivery lumen.

Example 38: The system of any example herein, in particular example 37, wherein a distal end of the delivery catheter is configured to be disposed within the first delivery lumen of the tapered access sheath while the delivery catheter is preloaded within the tapered access sheath.

Example 39: The system of any example herein, in particular example 38, wherein a distal end of the medical device is configured to be disposed distally of a distal end of the delivery catheter while the medical device is preloaded within the delivery catheter.

Example 40: The system of any example herein, in particular example 39, wherein the distal end of the medical device is proximal of and adjacent to a proximal edge of the side opening of the tapered access sheath while the medical device is preloaded within the delivery catheter.

Example 41: The system any example herein, in particular examples 37 to 40, wherein the recess comprises a width configured to slidably receive at least a portion of a guidewire, the first delivery lumen comprises a diameter configured to slidably receive at least a portion of the delivery catheter receiving the medical device, and while the delivery catheter receiving the medical device is deployed from the access sheath shaft, a portion of the delivery catheter is configured to remain disposed through the side opening.

Example 42: The system of any example herein, in particular example 41, wherein, while the delivery catheter receiving the medical device is deployed from the access sheath shaft, at least a portion of a distal portion of the delivery catheter receiving the medical device is configured to be disposed distally of a distal end of the access sheath shaft and to extend along a path offset from a longitudinal axis of the recess.

Example 43: The system of any example herein, in particular examples 37 to 40, wherein the recess comprises a width configured to accommodate a proximal portion of the delivery catheter receiving the medical device, the proximal portion of the delivery catheter receiving the medical device having a smaller diameter than a distal portion of the delivery catheter receiving the medical device, the first delivery lumen comprises a diameter configured to accommodate the distal portion of the delivery catheter receiving the medical device, and while the delivery catheter receiving the medical device is deployed from the access sheath shaft, at least a portion of the proximal portion of the delivery catheter receiving the medical device is configured to be slidably disposed through the recess.

Example 44: The system of any example herein, in particular example 43, wherein while the delivery catheter receiving the medical device is deployed from the access sheath shaft, at least a portion of the distal portion of the delivery catheter receiving the medical device is configured to be disposed distally of a distal end of the access sheath shaft and extend along a path coaxial with a longitudinal axis of the recess.

Example 45: The system of any example herein, in particular examples 37 to 44, wherein a longitudinal axis of the first delivery lumen is parallel to that of the recess.

Example 46: The system of any example herein, in particular examples 37 to 45, wherein longitudinal axes of the first delivery lumen and of the recess are parallel to a longitudinal axis of the access sheath shaft.

Example 47: The system of any example herein, in particular examples 37 to 46, wherein an orientation of the side opening is the same as that of the recess.

Example 48: The system of any example herein, in particular examples 37 to 47, wherein the side opening extends around a first partial circumferential portion of the access sheath shaft and the recess extends around a second partial circumferential portion of the access sheath shaft, the first and second partial circumferential portions each comprising at least a portion that overlaps with one another.

Example 49: The system of any example herein, in particular examples 37 to 48, further comprising a protrusion along an exterior surface of a sidewall portion of the access sheath shaft defining at least a portion of the side opening, the protrusion being configured to be pushed against an inner lumen wall of an expandable distal end portion of an introducer sheath while the tapered access sheath is slidably disposed through an introducer sheath delivery lumen and translated distally relative to the introducer sheath.

Example 50: The system of any example herein, in particular example 49, wherein the protrusion is along at least a portion of the sidewall portion defining a proximal edge of the side opening.

Example 51: The system of any example herein, in particular example 49 or 50, wherein the protrusion is along a sidewall portion defining a proximal edge of the side opening, the proximal edge of the side opening extending around a portion of a circumference of the access sheath shaft.

Depending on the example, certain acts, events, or functions of any of the processes or algorithms described herein can be performed in a different sequence, may be added, merged, or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without author input or prompting, whether these features, elements and/or steps are included or are to be performed in any particular example. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain examples require at least one of X, at least one of Y and at least one of Z to each be present.

It should be appreciated that in the above description of examples, various features are sometimes grouped together in a single example, Figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the inventions herein disclosed and claimed below should not be limited by the particular examples described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which examples belong. It be further understood that terms, such as those defined in commonly used dictionaries, should be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A tapered medical access sheath comprising:
an access sheath hub; and
an access sheath shaft extending distally from the access sheath hub, the access sheath shaft comprising:
a first delivery lumen extending from a proximal end of the access sheath shaft to a side opening on a distal portion of the access sheath shaft, the first delivery lumen having a diameter configured to slidably receive a distal portion of a medical device disposed around a guidewire;
a tapered distal end portion disposed distally of the side opening; and
a recess extending along a longitudinal dimension of the tapered distal end portion from a proximal end to a distal end of the tapered distal end portion, the recess being in fluid communication with the first delivery lumen and having a width smaller than the diameter of the first delivery lumen.

2. The sheath of claim 1, wherein:
an inner lumen wall portion defining a portion of the first delivery lumen adjacent to the side opening comprises a ramp configured to guide an exit trajectory of the distal portion of the medical device through the side opening; and
the recess extends from at least a portion of the ramp to a distal slit on a distal end of the access sheath shaft.

3. The sheath of claim 1, wherein:
the recess comprises a width configured to slidably receive at least a portion of a guidewire;
the first delivery lumen comprises a diameter configured to slidably receive at least a portion of a medical device, and the width of the recess being sized to provide a portion of the medical device remaining disposed through the side opening while the medical device is in a deployed state.

4. The sheath of claim 1, wherein:
the recess comprises a width configured to accommodate a proximal portion of a medical device, the proximal portion of the medical device having a smaller diameter than that of a distal portion of the medical device;
the first delivery lumen comprises a diameter configured to accommodate the distal portion of the medical device, and the width of the recess being sized to slidably receive the proximal portion of the medical device while the distal portion of the medical device is disposed distally of the access sheath shaft.

5. The sheath of claim 1, wherein the tapered distal end portion is distal of and adjacent to a distal edge of the side opening.

6. The sheath of claim 1, wherein a sidewall portion of the access sheath shaft defining a proximal edge of the side opening extends around a portion of a circumference of the distal portion of the access sheath shaft.

7. The sheath of claim 6, wherein the sidewall portion of the access sheath shaft defining a proximal edge of the side opening extends around a half of the circumference of the distal portion of the access sheath shaft.

8. The sheath of claim 1, further comprising a protrusion along an exterior surface of a sidewall portion of the access sheath shaft defining a proximal edge of the side opening, the protrusion being configured to be pushed against an inner lumen wall of an expandable distal end portion of an introducer sheath while the access sheath shaft is slidably disposed through an introducer sheath delivery lumen and translated distally relative to the introducer sheath.

9. The sheath of claim 1, wherein:
an inner lumen wall portion defining a portion of the first delivery lumen adjacent to the side opening comprises a ramp configured to guide an exit trajectory of a distal portion of a medical device through the side opening; and
a sidewall portion of the access sheath shaft defining a distal portion of the side opening extends along at least a portion of the ramp.

10. The sheath of claim 9, wherein the sidewall portion defining the distal portion of the side opening extends along respective edges of the ramp.

11. A method of delivering a medical device, the method comprising:
providing a tapered access sheath comprising an access sheath shaft having a tapered distal end portion, and the access sheath shaft further comprising:
a first delivery lumen extending from a proximal end to a side opening of the access sheath shaft; and
a recess extending through the tapered distal end portion and in fluid communication with the first delivery lumen, the recess having a width smaller than a diameter of the first delivery lumen;
providing a medical device comprising at least a portion disposed within the first delivery lumen;
providing a guidewire comprising corresponding portions disposed within a guidewire lumen of the medical device, the first delivery lumen and the recess of the access sheath shaft, and a bodily lumen;
advancing at least a portion of the access sheath shaft comprising the at least a portion of the medical device disposed therein along the guidewire into a bodily lumen; and
advancing the medical device distally relative to the access sheath shaft along the guidewire to position a distal portion of the medical device out of the first delivery lumen through the side opening and externally of the access sheath shaft, and to lift a corresponding portion of the guidewire out of the recess.

12. The method of claim 11, wherein advancing the medical device distally relative to the access sheath shaft along the guidewire comprises positioning at least a portion of the distal portion of the medical device disposed externally of the first delivery lumen along an axis offset from a longitudinal axis of the recess; and the method further comprising maintaining a portion of the medical device disposed through the side opening after advancing the portions of the medical device out of the first delivery lumen through the side opening.

13. The method of claim 11, wherein advancing the medical device distally relative to the access sheath shaft along the guidewire comprises:
advancing the medical device until a portion of the medical device fits within the recess;
positioning the portion of the medical device through the recess; and
disposing the distal portion of the medical device distally of a distal end of the access sheath shaft.

14. The method of claim 11, further comprising positioning an introducer sheath into the bodily lumen, and wherein advancing at least a portion of the access sheath shaft comprising the at least a portion of the medical device disposed therein along the guidewire into a bodily lumen comprises advancing the at least a portion of the access sheath shaft and medical device through a sheath delivery lumen of the introducer sheath positioned into the bodily lumen.

15. The method of claim 14, wherein advancing the at least a portion of the access sheath shaft and medical device through the introducer sheath comprises pushing a protrusion on an exterior surface of a side wall portion of the access sheath shaft defining a proximal edge of the side opening against an inner lumen wall portion of an expandable distal end portion of the introducer sheath to expand the expandable distal end portion.

16. A method of delivering a medical device, the method comprising:
providing a tapered access sheath comprising an access sheath shaft having a first delivery lumen extending through the access sheath shaft to a side opening on a distal portion of the access sheath shaft, and a recess extending from the first delivery lumen to a distal end of the access sheath shaft;
providing a medical device comprising a guidewire lumen extending therethrough, the medical device comprising a distal portion disposed within the first delivery lumen;
providing a guidewire comprising corresponding portions disposed within the guidewire lumen of the medical device, and the first delivery lumen and the recess of the access sheath shaft; and
advancing the medical device along the guidewire to deploy the distal portion of the medical device through the side opening.

17. The method of claim 16, wherein advancing the medical device to deploy the distal portion of the medical device comprises lifting a corresponding portion of the guidewire out of the recess.

18. The method of claim 16, wherein advancing the medical device to deploy the distal portion of the medical device comprises advancing the distal portion of the medical device through the side opening and maintaining a portion of the medical device extending through the side opening while the distal portion is in a deployed state.

19. The method of claim 16, wherein advancing the medical device to deploy the distal portion of the medical device comprises positioning a portion of the medical device through a distal slit at the distal end of the access sheath shaft to provide at least a portion of a proximal portion of the medical device slidably received within the recess and the distal portion of the medical device disposed distally of the distal end of the access sheath shaft.

20. The method of claim 19, further comprising positioning the distal portion of the medical device distally of the access sheath shaft and along a path coaxial with a longitudinal axis of the recess.
